(19) United States
(12) Patent Application Publication
(10) Pub. No.: US 2024/0245753 A1
(43) Pub. Date: Jul. 25, 2024

(54) METHODS OF REDUCING SIDE EFFECTS OF ANTI-CD30 ANTIBODY DRUG CONJUGATE THERAPY

(71) Applicant: SEAGEN INC., Bothell, WA (US)

(72) Inventors: Thomas Manley, Bothell, WA (US); Neil Josephson, Seattle, WA (US)

(21) Appl. No.: 18/449,591

(22) Filed: Aug. 14, 2023

Related U.S. Application Data
(63) Continuation of application No. 16/760,916, filed on May 1, 2020, now abandoned.
(60) Provisional application No. 62/739,635, filed on Oct. 1, 2018.
(60) Provisional application No. 62/580,261, filed on Nov. 1, 2017.

(51) Int. Cl. A61K 38/19; A61K 31/573; A61K 31/675; A61K 31/704; A61K 39/00; A61K 47/68; A61P 25/02; A61P 35/00

(52) U.S. Cl. CPC A61K 38/193; A61K 31/573; A61K 31/675; A61K 31/704; A61K 47/6817; A61K 47/6849; A61P 25/02; A61P 35/00; A61K 2039/545

(57) ABSTRACT

The present disclosure, relates, in general to methods for improving adverse events in subjects having a mature T cell lymphoma and who are receiving treatment with an anti-CD30 antibody drug conjugate in combination with accompanying chemotherapy. Adverse events include peripheral neuropathy and neutropenia.

### CROSS REFERENCE TO RELATED APPLICATIONS

The present application is a continuation of U.S. patent application Ser. No. 16/760,916, filed May 1, 2020, which is a national phase of PCT/US18/58613, filed Nov. 1, 2018 which claims the priority benefit of U.S. Provisional Patent Application No. 62/580,261, filed Nov. 1, 2017, and U.S. Provisional Patent Application No. 62/739,635, filed Oct. 1, 2018, each of which is incorporated herein by reference.

### INCORPORATION BY REFERENCE OF MATERIAL SUBMITTED ELECTRONICALLY

The Sequence Listing, which is a part of the present disclosure, is submitted concurrently with the specification as an XML file. The name of the XML file containing the Sequence Listing is “52570A_SeqListing.xml; Size: 16,129 bytes; Created: Oct. 31, 2023”. The subject matter of the Sequence Listing is incorporated herein in its entirety by reference.

### FIELD OF THE DISCLOSURE

The present disclosure relates, in general, to methods of reducing adverse effects, such as neutropenia and peripheral neuropathy in subjects having a Mature T cell lymphoma receiving anti-CD30 antibody drug conjugate therapy, optionally in combination with a chemotherapeutic regimen of cyclophosphamide, doxorubicin and prednisone.

### BACKGROUND

T-cell lymphomas are a subset of aggressive non-Hodgkin lymphomas (NHL) that comprise approximately 10-15% of all newly diagnosed cases of NHL in the United States. According to the 2008 World Health Organization (WHO) Classification schema, there are 18 subtypes of mature T- and natural killer (NK) cell neoplasms (Swerdlow 2008). Various subtypes of T- and NK-cell lymphomas are known to express the cell surface marker CD30; most notably, sALCL, in which CD30 expression is a hallmark of the diagnosis (Savage 2008).

CD30-positive mature T-cell lymphomas, including sALCL, peripheral T-cell lymphoma—not otherwise specified (PTCL-NOS), angioimmunoblastic T-cell lymphoma (AITL) and others, are aggressive lymphoid neoplasms that often present with advanced stage, symptomatic disease. These difficult-to-treat lymphomas are often grouped together for enrollment in clinical trials based on their universally dismal outcomes. Five-year overall survival (OS) in the over 1,300-patient International Peripheral T-Cell and Natural Killer/T-Cell Lymphoma Study was poor and ranged from 12 to 49% depending on histologic subtype (Vose 2008). Five-year failure-free survival, defined as time from initial diagnosis to progression, relapse after response, or death resulting from any cause, ranged from 6 to 36%. Other studies have reported CR rates to cyclophosphamide, doxorubicin, vincristine, and prednisone (CHOP) therapy between 40-50% (Mercadal 2008; Simon 2010). These data confirm 2 distinct unmet needs. First, there is a failure to induce a high rate of initial complete remissions (CRs), and second, those patients who do respond to combination chemotherapy experience disease progression at an unacceptably high rate. Increasing the proportion of patients achieving and maintaining CRs may result in a clinically meaningful improvement in progression-free survival (PFS) and OS.

Frontline treatment of mature T-cell and NK-cell neoplasms is dependent on the subtype of disease and often includes clinical trials as the preferred therapeutic option (NCCN 2013). For most subtypes, anthracycline-based multiagent chemotherapy regimens such as CHOP are commonly utilized. The notable exception is extranodal NK/T-cell lymphoma, nasal and non-nasal types, where concurrent chemoradiotherapy regimens are employed.

Although no randomized studies have been conducted to establish the use of CHOP in patients with CD30-positive mature T-cell neoplasms, it is the most commonly used regimen in the frontline treatment of these patients. The International Peripheral T-Cell and Natural Killer/T-Cell Lymphoma Study results indicate that over 85% of patients were treated with an anthracycline-based multiagent chemotherapy regimen (Vose 2008). In published studies that have compared new treatment approaches to an established standard of care, CHOP administered every 3 weeks (CHOP-21) has been used as the control arm (Simon 2010). In addition, published guidelines recommend enrollment into a clinical trial or CHOP as appropriate frontline treatment options for patients with a diagnosis of “peripheral T-cell lymphoma, noncutaneous” (NCCN 2013). Guidelines support administration of 6 cycles of CHOP therapy for patients with Stage I-II disease and International Prognostic Index (IPI) score of 0-2, and 6-8 cycles of CHOP therapy for Stage I-II patients with an IPI score of 3-5 and all Stage III-IV patients (Schmitz 2010; NCCN 2013). Comparison of non-randomized clinical trials does not support a difference in activity between 6 or 8 cycles of CHOP, with 6-8 cycles commonly employed in clinical practice (Coiffier 2002; Schmitz 2010). As mentioned above, the response to CHOP chemotherapy is suboptimal with CR rates ranging from approximately 40-50%, and overall response rates of approximately 75% (Mercadal 2008; Simon 2010; Dearden 2011). The estimates of long-term outcome in the mature T-cell lymphoma population, regardless of the backbone of anthracycline-based multiagent chemotherapy, are suboptimal with a median EFS or PFS of 12-18 months and a median OS of less than 4 years (depending on histologic subtype and IPI score).

The high rate of subsequent disease progression among patients responding to frontline therapy led some researchers to employ autologous stem cell transplant (SCT) as a means of improving long-term outcomes; however, no randomized studies have been conducted. National and international guidelines support observation, a clinical trial, or the use of autologous SCT as acceptable options for patients who achieve a CR following frontline therapy (Dearden 2011; NCCN 2013).

The clinical safety and activity of brentuximab vedotin 1.8 mg/kg administered every 3 weeks were evaluated in a pivotal phase 2 study of patients with relapsed or refractory sALCL (Study SG035-0004). In this study, all patients had previously received at least 1 prior regimen of multiagent systemic chemotherapy with curative intent. The majority of patients had a diagnosis of ALK-negative disease (72%); relative to the most recent therapy, 50% of patients were refractory. Additionally, approximately 60% of patients had primary refractory disease, defined as failure to achieve a complete remission (CR) with frontline therapy or progression within 3 months of completing frontline therapy, and 22% of patients had never achieved a response with any previous therapy. In this study of highly refractory patients, the objective response (CR+PR) rate was 86%, with 57% of patients achieving a CR (Pro 2012). The median duration of response was 12.6 months, and in the subset of patients who achieved a CR, the median duration of response was 13.2 months. Brentuximab vedotin was generally well tolerated, with manageable side effects.

### SUMMARY

The present disclosure provides improved methods for administering an anti-CD30 antibody-drug conjugate and reducing adverse events in subject having a mature T cell lymphoma and receiving anti-CD30 antibody drug conjugate therapy. In some embodiments, the anti-CD30 antibody-drug conjugate is administered in combination with a chemotherapy regimen. It is contemplated that the therapeutic regimen may include chemotherapeutics known in the field of cancer treatment. Exemplary chemotherapeutics are disclosed in greater detail in the Detailed Description. In various embodiments, the methods herein include treatment comprising a chemotherapy consisting essentially of cyclophosphamide, doxorubicin and prednisone (CHP). In some embodiments, side effects such as peripheral neuropathy are reduced by adjusting the amount and/or timing of anti-CD30 antibody drug conjugate. In other embodiments, side effects including neutropenia, febrile neutropenia or infection are reduced by co-administration of the anti-CD30 antibody drug conjugate with a granulopoiesis stimulating factor.

In one aspect, the disclosure provides a method of administering an anti-CD30 drug conjugate, e.g., brentuximab vedotin, to a subject having a mature T cell lymphoma at a dose of 1.8 mg/kg, administered, e.g., every three weeks. The mature T cell lymphoma may be more particularly diagnosed as, for example, peripheral T cell lymphoma (PTCL), PTCL entities typically manifesting as nodal involvement, angioimmunoblastic T-cell lymphoma, anaplastic large cell lymphomas, peripheral T-cell lymphoma-not otherwise specified, subcutaneous panniculitis-like T-cell lymphoma, hepatosplenic gamma delta T-cell lymphoma, enteropathy-type intestinal T-cell lymphoma, and extranodal T-cell lymphoma-nasal type.

In various embodiments, the disclosure provides a method for treating a subject having a mature T cell lymphoma that has exhibited Grade 2 or greater peripheral neuropathy after starting treatment with a combination therapy comprising an anti-CD30 antibody drug conjugate at a dose of 1.8 mg/kg in combination with a chemotherapy consisting essentially of cyclophosphamide, doxorubicin and prednisone (CHP) every three weeks, comprising administering anti-CD30 antibody drug conjugate at a dose of 0.9 mg/kg to 1.2 mg/kg. In various embodiments, the subject exhibits Grade 2 or Grade 3 peripheral neuropathy.

In various embodiments, when the subject exhibits Grade 3 neuropathy, the administration of anti-CD30 antibody drug conjugate is withheld until peripheral neuropathy decreases to Grade 2 or less and then 0.9 mg/kg to 1.2 mg/kg anti-CD30 antibody drug conjugate is administered. In various embodiments, when the subject exhibits Grade 3 neuropathy, the administration of anti-CD30 antibody drug conjugate is reduced, optionally to 0.9 to 1.2 mg/kg, until peripheral neuropathy decreases to Grade 2 or less and then 0.9 mg/kg to 1.2 mg/kg anti-CD30 antibody drug conjugate is administered or maintained.

In various embodiments, the subject exhibited Grade 2 or 3 peripheral neuropathy after starting anti-CD30 antibody drug conjugate therapy at a dose of 1.8 mg/kg in combination with a chemotherapy consisting essentially of cyclophosphamide (C), doxorubicin (H) and prednisone (P) therapy, preferably the anti-CD30 antibody drug conjugate is brentuximab vedotin, administered every three weeks.

In various embodiments, the dose of anti-CD30 antibody drug conjugate is increased from 0.9 to 1.2 mg/kg to 1.8 mg/kg or 1.2 mg/kg after the Grade 2 or Grade 3 peripheral neuropathy improves to Grade 1 or less. In various embodiments, when the anti-CD30 antibody drug conjugate administration is at 1.2 mg/kg, the administration may be every two weeks, up to a maximum of 120 mg every two weeks.

In various embodiments, the disclosure provides a method for treating a subject having a mature T cell lymphoma that has exhibited Grade 2 or greater peripheral neuropathy after starting treatment with a therapy comprising an anti-CD30 antibody drug conjugate at a dose of 1.8 mg/kg, comprising administering anti-CD30 antibody drug conjugate at a dose of 0.9 mg/mg to 1.2 mg/kg. In various embodiments, the subject exhibits Grade 2 or Grade 3 peripheral neuropathy.

In various embodiments, when the subject exhibits Grade 3 neuropathy, the administration of anti-CD30 antibody drug conjugate is withheld until peripheral neuropathy decreases to Grade 2 or less and then 0.9 mg/kg to 1.2 mg/kg anti-CD30 antibody drug conjugate is administered. In various embodiments, when the subject exhibits Grade 3 neuropathy, the administration of anti-CD30 antibody drug conjugate is reduced, optionally to 0.9 to 1.2 mg/kg, until peripheral neuropathy decreases to Grade 2 or less and then 0.9 mg/kg to 1.2 mg/kg anti-CD30 antibody drug conjugate is administered.

In various embodiments, the dose of anti-CD30 antibody drug conjugate is increased from 0.9 to 1.2 mg/kg to 1.8 mg/kg or 1.2 mg/kg after the Grade 2 or Grade 3 peripheral neuropathy improves to Grade 1 or less, wherein if the dose is increased to 1.8 mg/kg, the administration optionally is in combination with a chemotherapy consisting essentially of cyclophosphamide, doxorubicin and prednisone therapy, preferably the anti-CD30 antibody drug conjugate is brentuximab vedotin, administered every three weeks.

In various embodiments, the mature T cell lymphoma is peripheral T cell lymphoma (PTCL). In various embodiments, when the mature T cell lymphoma is PTCL, and wherein if the subject is diagnosed with Grade 2 or greater peripheral motor neuropathy after starting treatment with a combination therapy comprising an anti-CD30 antibody drug conjugate at a dose of 1.8 mg/kg every three weeks in combination with a chemotherapy consisting essentially of cyclophosphamide, doxorubicin and prednisone (CHP), the dose of anti-CD30 antibody drug conjugate is reduced to 1.2 mg/kg.

In various embodiments, when the mature T cell lymphoma is peripheral T cell lymphoma, and wherein if the subject is diagnosed with Grade 3 or greater peripheral sensory neuropathy after starting treatment with a combination therapy comprising an anti-CD30 antibody drug conjugate at a dose of 1.8 mg/kg every three weeks in combination with a chemotherapy consisting essentially of cyclophosphamide, doxorubicin and prednisone (CHP), the dose of anti-CD30 antibody drug conjugate is reduced to 1.2 mg/kg.

In various embodiments, the PTCL is selected from the group consisting of systemic anaplastic large cell lymphoma (sALCL), angioimmunoblastic T-cell lymphoma (AITL), peripheral T-cell lymphoma-not otherwise specified (PTCL-NOS), Adult T-Cell Leukemia/Lymphoma (ATLL), Enteropathy-associated T-cell lymphoma (EATL) and Hepatosplenic T-cell lymphoma.

In various embodiments, the PTCL is a sALCL. In various embodiments, the sALCL is selected from the group consisting of anaplastic lymphoma kinase positive (ALK+) sALCL and anaplastic lymphoma kinase negative (ALK−) sALCL. In various embodiments, the sALCL is an ALK+sALCL. In various embodiments, the sALCL is an ALK−sALCL.

In various embodiments, the PTCL is not a sALCL. In various embodiments, the PTCL is selected from the group consisting of angioimmunoblastic T-cell lymphoma (AITL), peripheral T-cell lymphoma-not otherwise specified (PTCL-NOS), Adult T-Cell Leukemia/Lymphoma (ATLL), Enteropathy-associated T-cell lymphoma (EATL) and Hepatosplenic T-cell lymphoma.

In various embodiments, the PTCL is not an AITL. In various embodiments, the PTCL is selected from the group consisting of systemic anaplastic large cell lymphoma (sALCL), peripheral T-cell lymphoma-not otherwise specified (PTCL-NOS), Adult T-Cell Leukemia/Lymphoma (ATLL), Enteropathy-associated T-cell lymphoma (EATL) and Hepatosplenic T-cell lymphoma.

In various embodiments, the subject has an International Prognostic Index (IPI) score of 0 or 1. In various embodiments, the subject has an International Prognostic Index (IPI) score 2. In various embodiments, the subject has an International Prognostic Index (IPI) score of 2 or 3. In various embodiments, the subject has an International Prognostic Index (IPI) score 4. In various embodiments, the subject has an International Prognostic Index (IPI) score of 4 or 5.

In various embodiments, the subject has a Baseline ECOG Status of 0 or 1. In various embodiments, the subject has a Baseline ECOG Status of 2.

In various embodiments, the subject is newly diagnosed with PTCL and/or has not previously been treated for a hematologic cancer. In various embodiments, the subject has previously been treated for a hematologic cancer. In various embodiments, the cancer has relapsed or is refractory.

In various embodiments, the PTCL is a stage Ill or stage IV PTCL.

In various embodiments, the PTCL is a CD30-expressing PTCL tumor. In various embodiments, the PTCL is a CD30-expressing PTCL and the CD30 expression is 10% of lymphoma cells.

In various embodiments, the CD30 expression is measured by a FDA approved test. Exemplary tests include local pathology assessment in a CD30-qualified laboratory; CD30 positivity confirmed in diagnostic biopsy using immunohistochemistry. The 3 following criteria are used to determine CD30 positivity:
1) CD30 detected in 10% or greater of neoplastic cells (in cases where enumeration of neoplastic cells was not possible, total lymphocytes may have been used).
2) CD30 staining at any intensity above background, and
3) Membranous, cytoplasmic, and/or golgi pattern of expression of the CD30 antigen.

In various embodiments, the neuropathy is measured periodically using standard assays known in the art.

In various embodiments, doses of anti-CD30 antibody drug conjugate may be reduced if the patient experiences renal or hepatic impairment. In various embodiments, if the subject experiences mild hepatic impairment (Child-Pugh A) the dose is reduced to approximately 1.2 mg/kg and is administered every 2 weeks, up to a maximum of 120 mg (depending on weight of patient) administered every 2 weeks.

In various embodiments, if the anti-CD30 antibody drug conjugate (ADC) is administered at 1.8 mg/kg with CHP combination therapy, the combination therapy is administered every three weeks. In various embodiments, the combination therapy is administered on day 1 of a 21 day cycle. In various embodiments, the ADC+CHP combination therapy is administered for no more than eight cycles. In various embodiments, the ADC+CHP combination therapy is administered for six to eight cycles. In various embodiments, the A+CHP therapy is administered for 4, 5, 6, 7 or 8 cycles. Optionally, the subject receives single-agent anti-CD30 antibody drug conjugate, e.g., brentuximab vedotin, for eight to 10 additional cycles for a total of 16 cycles.

In various embodiments, the ADC or combination therapy is administered until a PET scan determines there is no tumor or progression of tumor.

In various embodiments, the neuropathy is peripheral motor neuropathy or peripheral sensory neuropathy. In various embodiments, the ADC or combination therapy reduces one or more symptoms of peripheral neuropathy selected from the group consisting of paresthesia, hypoesthesia, polyneuropathy, muscular weakness, and demyelinating polyneuropathy.

In various embodiments, the dose of anti-CD30 antibody drug conjugate is delayed by one week or two weeks if peripheral neuropathy appears, and ADC or combination therapy is continued when the neuropathy is resolved or determined to be Grade 2 or less, or Grade 1 or less.

In a second aspect, the disclosure provides a method for treating a mature T cell lymphoma in a subject comprising co-administering an anti-CD30 antibody drug conjugate, optionally with a chemotherapy, with a granulopoiesis stimulating factor at the initiation of, or first administration of, the antibody drug conjugate therapy, e.g. as primary prophylaxis. In various embodiments, the granulopoiesis stimulating factor can be used also in combination with any standard or modified chemotherapeutic regimen, e.g., as a frontline therapy. For example, treatment at initiation of therapy, e.g., as primary prophylaxis, includes wherein the granulopoiesis stimulating factor is administered from within 1 day to within 7 days after the initiation of or first administration of the therapy, e.g., ADC or combination therapy. In various embodiments, the granulopoiesis stimulating factor is administered from within 2 days to within 5 days after the initiation of or first administration of the therapy, e.g., ADC or combination therapy. In some embodiments, the granulopoiesis stimulating factor is administered on the same day as the ADC or combination therapy.

In various embodiments, provided herein is a method for treating a mature T cell lymphoma in a subject comprising administering a combination therapy comprising an anti-CD30 antibody drug conjugate in combination with a chemotherapy consisting essentially of cyclophosphamide, doxorubicin and prednisone (CHP) and prophylactically administering a granulopoiesis stimulating factor, wherein the granulopoiesis stimulating factor is administered with the initiation of the combination therapy.

In various embodiments of this second aspect, the method is for reducing the incidence of neutropenia or febrile neutropenia in a subject having mature T cell lymphoma and receiving therapy comprising anti-CD30 antibody drug conjugate, optionally in combination with a chemotherapy. In various embodiments, the disclosure provides a method for reducing the incidence of neutropenia in a subject having mature T cell lymphoma and receiving a combination therapy comprising an anti-CD30 antibody drug conjugate in combination with a chemotherapy consisting essentially of cyclophosphamide, doxorubicin and prednisone (CHP) comprising administering to the subject a granulopoiesis stimulating factor, wherein the granulopoiesis stimulating factor is administered with initiation of the combination therapy.

In various embodiments of this second aspect, the method is for decreasing the incidence of infection, or for decreasing the incidence of other adverse events, in a subject having mature T cell lymphoma and receiving therapy comprising anti-CD30 antibody drug conjugate, optionally in combination with a chemotherapy. In various embodiments, the disclosure provides a method for decreasing the incidence of infection in a subject having mature T cell lymphoma and receiving a combination therapy comprising an anti-CD30 antibody drug conjugate in combination with a chemotherapy consisting essentially of cyclophosphamide, doxorubicin and prednisone (CHP) comprising administering to the subject a granulopoiesis stimulating factor in an amount effective to reduce infections, wherein the granulopoiesis stimulating factor is administered with the initiation of the combination therapy.

In various embodiments, it is contemplated that the anti-CD30 antibody drug conjugate is brentuximab vedotin.

In various embodiments, the granulopoiesis stimulating factor is administered from 1 day to 7 days, or from 1 day to 5 days, or from 2 days to 5 days, after a second or subsequent, administration of the therapy. In some embodiments, the granulopoiesis stimulating factor is administered on the same day as the second or subsequent administration of the ADC or combination therapy.

In various embodiments, the granulopoiesis stimulating factor is administered to a subject that has not received an anti-CD30 antibody drug conjugate therapy previously, or to a subject before the subject has experienced treatment-emergent neutropenia. In various embodiments, the subject has not experienced treatment-emergent grade 3-4 neutropenia after administration of the ADC or combination therapy.

In various embodiments, the granulopoiesis stimulating factor is granulocyte colony stimulating factor (GCSF). In various embodiments, the GCSF is a long-acting GCSF or is not long-acting GCSF. In various embodiments, the granulopoiesis stimulating factor is granulocyte monocyte colony stimulating factor (GM-CSF). In various embodiments, the GCSF is long-acting, and is administered in a single dose 1, 2 or 3 days after initiation of the ADC or combination therapy. In various embodiments, the stimulating factor is GMCSF, or the GCSF is not long acting, and is administered in multiple doses (e.g. multiple daily doses) starting at 1, 2, 3, 4, 5, 6, or 7 days after the initiation of the therapy for a duration of at least 3, 4, 5, 6, 7, 8, 9, 10, 11, 12 or more days. In various embodiments, the granulopoiesis stimulating factor is pegfilgrastim or filgrastim.

In various embodiments, the anti-CD30 antibody drug conjugate, optionally in combination with a chemotherapy consisting essentially of cyclophosphamide, doxorubicin and prednisone (CHP), is administered every 3 weeks.

In various embodiments, anti-CD30 antibody drug conjugate is administered on day 1 of a 21-day cycle. In various embodiments, the method further comprises administering a chemotherapy consisting essentially of cyclophosphamide, doxorubicin and prednisone (CHP) as a combination therapy, preferably the anti-CD30 antibody drug conjugate is brentuximab vedotin, on the same day as the anti-CD30 antibody drug conjugate.

In various embodiments, the anti-CD30 antibody of the anti-CD30 antibody drug conjugate comprises i) a heavy chain CDR1 set out in SEQ ID NO: 4, a heavy chain CDR2 set out in SEQ ID NO: 6, a heavy chain CDR3 set out in SEQ ID NO: 8; and ii) a light chain CDR1 set out in SEQ ID NO: 12, a light chain CDR2 set out in SEQ ID NO: 14, and a light chain CDR13 set out in SEQ ID NO: 16.

In various embodiments, the anti-CD30 antibody of the anti-CD30 antibody drug conjugate also comprises i) an amino acid sequence at least 85% identical to a heavy chain variable region set out in SEQ ID NO: 2 and ii) an amino acid sequence at least 85% identical to a light chain variable region set out in SEQ ID NO: 10. It is contemplated that the amino acid variable region sequence can be 90%, 95%, 96% 97%, 98% or 99% identical to either SEQ ID NO:2 or SEQ ID NO: 10.

In various embodiments, the anti-CD30 antibody of the anti-CD30 antibody drug conjugate is a monoclonal anti-CD30 antibody. In various embodiments, the anti-CD30 antibody of the anti-CD30 antibody drug conjugate is a chimeric AC10 antibody.

In various embodiments, the antibody drug conjugate comprises monomethyl auristatin E and a protease-cleavable linker. In various embodiments, the protease cleavable linker is comprises a thiolreactive spacer and a dipeptide. In various embodiments, the protease cleavable linker consists of a thiolreactive maleimidocaproyl spacer, a valine-citrulline dipeptide, and a p-amino-benzyloxycarbonyl spacer.

In various embodiments, the antibody is an IgG antibody, preferably an IgG1 antibody.

In various embodiments, the anti-CD30 antibody drug conjugate is brentuximab vedotin.

In various embodiments, the subject is also receiving a chemotherapy consisting essentially of cyclophosphamide, doxorubicin and prednisone (CHP) as a combination therapy. In various embodiments, the cyclophosphamide is administered at 750 mg/m², doxorubicin is administered at 50 mg/m², and prednisone is administered at 100 mg on days 1 to 5 of a 21 day cycle.

In various embodiments, the anti-CD30 antibody drug conjugate is brentuximab vedotin and is administered at 1.8 mg/kg, cyclophosphamide is administered at 750 mg/m², doxorubicin is administered at 50 mg/m², and prednisone is administered at 100 mg on days 1 to 5 of a 21 day cycle.

In various embodiments, the granulopoiesis stimulating factor, e.g., G-CSF, is administered in a dose range from 5 to 10 mcg/kg/day, or 300 to 600 mcg/day. In various embodiments, the granulopoiesis stimulating factor is administered at a dose of 6 mg/dose.

In various embodiments, the granulopoiesis stimulating factor is given intravenously or subcutaneously. In various embodiments, the granulopoiesis stimulating factor is given in a single dose or multiple doses, for example, a long-acting GCSF may be administered in a single dose or multiple doses on the same day, and a non-long-acting GCSF may be given in multiple doses over multiple days.

In any of the aspects disclosed herein, the subject is suffering from a mature T cell lymphoma (MTCL) selected from the group consisting of peripheral T cell lymphoma (PTCL), PTCL entities typically manifesting as nodal involvement, angioimmunoblastic T-cell lymphoma, anaplastic large cell lymphomas, peripheral T-cell lymphoma-not otherwise specified, subcutaneous panniculitis-like T-cell lymphoma, hepatosplenic gamma delta T-cell lymphoma, enteropathy-type intestinal T-cell lymphoma, and extranodal T-cell lymphoma-nasal type.

In various embodiments, the mature T cell lymphoma is peripheral T cell lymphoma (PTCL). In various embodiments, the PTCL is selected from the group consisting of systemic anaplastic large cell lymphoma (sALCL), angioimmunoblastic T-cell lymphoma (AITL), peripheral T-cell lymphoma-not otherwise specified (PTCL-NOS), Adult T-Cell Leukemia/Lymphoma (ATLL), Enteropathy-associated T-cell lymphoma (EATL) and Hepatosplenic T-cell lymphoma.

In various embodiments, the PTCL is a sALCL. In various embodiments, the sALCL is selected from the group consisting of anaplastic lymphoma kinase positive (ALK+) sALCL and anaplastic lymphoma kinase negative (ALK−) sALCL. In various embodiments, the sALCL is an ALK+sALCL. In various embodiments, the sALCL is an ALK−sALCL.

In various embodiments, the PTCL is not a sALCL. In various embodiments, the PTCL is selected from the group consisting of angioimmunoblastic T-cell lymphoma (AITL), peripheral T-cell lymphoma-not otherwise specified (PTCL-NOS), Adult T-Cell Leukemia/Lymphoma (ATLL), Enteropathy-associated T-cell lymphoma (EATL) and Hepatosplenic T-cell lymphoma.

In various embodiments, the PTCL is not an AITL. In various embodiments, the PTCL is selected from the group consisting of systemic anaplastic large cell lymphoma (sALCL), peripheral T-cell lymphoma-not otherwise specified (PTCL-NOS), Adult T-Cell Leukemia/Lymphoma (ATLL), Enteropathy-associated T-cell lymphoma (EATL) and Hepatosplenic T-cell lymphoma.

In various embodiments, the subject has an International Prognostic Index (IPI) score of 0 or 1. In various embodiments, the subject has an International Prognostic Index (IPI) score 2. In various embodiments, the subject has an International Prognostic Index (IPI) score of 2 or 3. In various embodiments, the subject has an International Prognostic Index (IPI) score 4. In various embodiments, the subject has an International Prognostic Index (IPI) score of 4 or 5.

In various embodiments, the subject has a Baseline ECOG Status of 0 or 1. In various embodiments, the subject has a Baseline ECOG Status of 2.

In various embodiments, the subject is newly diagnosed with PTCL and/or has not previously been treated for a hematologic cancer. In various embodiments, the subject has previously been treated for a hematologic cancer. In various embodiments, the cancer has relapsed or is refractory.

In various embodiments, the PTCL is a stage Ill or stage IV PTCL.

In various embodiments, the PTCL is a CD30-expressing PTCL tumor. In various embodiments, the PTCL is a CD30-expressing PTCL and the CD30 expression is 10% of lymphoma cells.

In various embodiments, the CD30 expression is measured by a FDA approved test. Exemplary tests include local pathology assessment in a CD30-qualified laboratory; CD30 positivity confirmed in diagnostic biopsy using immunohistochemistry. The 3 following criteria are used to determine CD30 positivity:
1) CD30 detected in 10% or greater of neoplastic cells (in cases where enumeration of neoplastic cells was not possible, total lymphocytes may have been used).
2) CD30 staining at any intensity above background, and
3) Membranous, cytoplasmic, and/or golgi pattern of expression of the CD30 antigen.

In various embodiments, when the lymphoma is peripheral T cell lymphoma, the granulopoiesis stimulating factor can be administered from 1 to 8 days after anti-CD30 antibody drug conjugate administration.

In a third aspect, the disclosure provides a method of treating a subject having mature T cell lymphoma comprising administering as frontline treatment an effective amount of a composition comprising bretuximab vedotin (A) in combination with chemotherapy consisting of cyclophosphamide, doxorubicin and prednisone (CHP), wherein the brentuximab vedotin is administered at 1.8 mg/kg every two weeks, cyclophosphamide is administered at 750 mg/m² on day 1 of a 21 day cycle, doxorubicin is administered at 50 mg/m² on day 1 of a 21 day cycle, and prednisone is administered at 100 mg on days 1 to 5 of a 21 day cycle, until a maximum of eight cycles, and wherein the brentuximab vedotin is administered within about 1 hour after administration of the CHP therapy; optionally the subject is characterized by one or more of the following: (1) ALK-positive sALCL with an IPI score greater than or equal to 2, ALK-negative sALCL, PTCL-NOS, AITL, Adult T-cell leukemia/lymphoma (ATLL; acute and lymphoma types only, must be positive for human T-cell leukemia virus 1), Enteropathy-associated T-cell lymphoma (EATL), Hepatosplenic T-cell lymphoma; (2) Fluorodeoxyglucose (FDG)-avid disease by PET and measurable disease of at least 1.5 cm by CT, or (3) an Eastern Cooperative Oncology Group (ECOG) performance status prior to therapy of 2 or less. The methods herein further provide that progression free survival (PFS) of the subject after therapy is maintained for greater than 1 year. In various embodiments, the progression free survival (PFS) of the subject after therapy is maintained for approximately 2 years. In certain embodiments, after six to eight cycles of A+CHP therapy the subject has a Deauville score of 3 or less, or 2 or less.

In another aspect, the disclosure provides an anti-CD30 antibody drug conjugate for use in treating a subject that has exhibited Grade 2 or greater peripheral neuropathy after starting treatment with a combination therapy comprising an anti-CD30 antibody drug conjugate at a dose of 1.8 mg/kg in combination with a chemotherapy consisting essentially of cyclophosphamide, doxorubicin and prednisone (CHP) every three weeks, wherein said patient is administered anti-CD30 antibody drug conjugate at a dose of 0.9 mg/kg to 1.2 mg/kg.

In a further aspect, contemplated herein is an anti-CD30 antibody drug conjugate for use in treating a mature T cell lymphoma in a subject comprising administering a combination therapy comprising an anti-CD30 antibody drug conjugate at a dose of 1.8 mg/kg in combination with a chemotherapy consisting essentially of cyclophosphamide, doxorubicin and prednisone (CHP) every three weeks and prophylactically administering a granulopoiesis stimulating factor, wherein the stimulating factor is administered with the initiation of the combination therapy, e.g., from 1 day to 7 days after the initiation of the combination therapy.

In a related aspect, also contemplated is an anti-CD30 antibody drug conjugate for use in reducing the incidence of neutropenia, infection or other adverse events in a subject comprising administering a combination therapy comprising an anti-CD30 antibody drug conjugate at a dose of 1.8 mg/kg in combination with a chemotherapy consisting essentially of cyclophosphamide, doxorubicin and prednisone (CHP) every three weeks and prophylactically administering a granulopoiesis stimulating factor, wherein the stimulating factor is administered with the initiation of the combination therapy, e.g., from 1 day to 7 days after the initiation of the combination therapy. In various embodiments, when the mature T cell lymphoma is PTCL, the granulopoiesis stimulating factor is administered from 1 to 8 days after initiation of the combination therapy.

It is specifically provided herein that all aspects of the disclosure described above with the methods of treatment are applicable to the anti-CD30 antibody drug conjugate for use in any of the indications described above.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### DETAILED DESCRIPTION

The present disclosure provides methods for improving adverse events associated with treatment of mature T Cell Lymphomas with an anti-CD30 antibody drug conjugate, optionally in combination with a chemotherapeutic regimen. The regimens described herein are effective for reducing peripheral neuropathy in treated patients as well as improving incidence of neutropenia, and/or febrile neutropenia, and/or infection associated with therapy.

### Definitions

Unless otherwise defined, all technical and scientific terms used herein have the same meaning as commonly understood by one of ordinary skill in the art to which this disclosure belongs. The following references provide one of skill with a general definition of many of the terms used in this disclosure: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

Each publication, patent application, patent, and other reference cited herein is incorporated by reference in its entirety to the extent that it is not inconsistent with the present disclosure.

As used herein and in the appended claims, the singular forms “a,” “and,” and “the” include plural referents unless the context clearly dictates otherwise. Thus, for example, reference to “a derivative” includes a plurality of such derivatives and reference to “a subject” includes reference to one or more subjects and so forth.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

“Therapeutically effective amount” as used herein refers to that amount of an agent effective to produce the intended beneficial effect on health.

A “therapy” as used herein refers to either single agent therapy with anti-CD30 antibody drug conjugate or a combination therapy comprising anti-CD30 drug conjugate in combination with a chemotherapeutic regimen. A preferred embodiment includes combination therapy comprising administering an anti-CD30 antibody drug conjugate with a chemotherapy consisting essentially of cyclophosphamide, doxorubicin and prednisone (CHP therapy).

“Antibody+CHP therapy”, or “A+CHP therapy” as used herein refers to treatment of a subject with an anti-CD30 antibody drug conjugate as described herein in combination with chemotherapy consisting essentially of cyclophosphamide, doxorubicin and prednisone therapy (CHP therapy).

“Lymphoma” as used herein is hematological malignancy that usually develops from hyper-proliferating cells of lymphoid origin. Lymphomas are sometimes classified into two major types: Hodgkin lymphoma (HL) and non-Hodgkin lymphoma (NHL). Lymphomas may also be classified according to the normal cell type that most resemble the cancer cells in accordance with phenotypic, molecular or cytogenic markers. Lymphoma subtypes under that classification include without limitation mature B-cell neoplasms, mature T cell and natural killer (NK) cell neoplasms, Hodgkin lymphoma and immunodeficiency-associated lympho-proliferative disorders. Lymphoma subtypes include precursor T-cell lymphoblastic lymphoma (sometimes referred to as a lymphoblastic leukemia since the T-cell lymphoblasts are produced in the bone marrow), follicular lymphoma, diffuse large B cell lymphoma, mantle cell lymphoma, B-cell chronic lymphocytic lymphoma (sometimes referred to as a leukemia due to peripheral blood involvement), MALT lymphoma, Burkitt® lymphoma, mycosis fungoides and its more aggressive variant Sezary® disease, peripheral T-cell lymphomas not otherwise specified, nodular sclerosis of Hodgkin lymphoma, and mixed-cellularity subtype of Hodgkin lymphoma.

“Peripheral T Cell lymphoma” refers to a subset of heterogeneous, aggressive non-Hodgkin lymphoma (NHL). As used herein “peripheral” does not refer to the extremities, but identifies PTCL as a cancer that arises in the lymphoid tissues outside of the bone marrow, such as lymph nodes, spleen, gastrointestinal tract, and skin (e.g., cutaneous peripheral T cell lymphoma). (Taken from lymphoma research foundation https://www.lymphoma.org/aboutlymphoma/nhl/ptcl/ PTCL can include involvement of T cells and natural killer (NK) cells. PTCL are different than cutaneous T cell lymphoma (CTCL), which originate in the skin. Peripheral T cell lymphoma includes systemic anaplastic large cell lymphoma (sALCL), angioimmunoblastic T-cell lymphoma (AITL), peripheral T-cell lymphoma-not otherwise specified (PTCL-NOS), Adult T-Cell Leukemia/Lymphoma (ATLL), Enteropathy-associated T-cell lymphoma (EATL) and Hepatosplenic T-cell lymphoma.

| | | CD30- | CD30- | CD30- |
|---|---|---|---|---|
| | | Expression | Expression | Expression |
| PTCL sub- | Total | at 10% | at 5% | at 1% |
| type | Patients^{1,2,3} | Threshold⁴ | Threshold⁴* | Threshold⁵ |
| ALCL | ~1950 | 100% | 100% | 100% |
| PTCL-NOS | ~2300 | 52% | 58% | Insufficient |
| AITL | ~1700 | 50% | 63% | Data |
| ATLL | ~450 | 53% | 56% | |
| EATL | ~200 | 50% | 50% | |
| Total | ~6,600 | ~4,200 | ~4,700 | |
| | | | (+12%) | |

¹SEER: https://seer.cancer.gov/statfacts/html/nhl.html Projected number of new NHL cases in 2018: 74,680
²Blood: http://www.bloodjournal.org/content/89/11/3909.long?sso-checked=true: PTCL accounts for 12% of NHL malignancies
³Annals of Oncology: https://www.ncbi.nlm.nih.gov/pmc/articles/PMC4481543/: Subtypes by percentage
⁴Blood: http://www.bloodjournal.org/cgi/pmidlookup?view=long&pmid=25224410: CD30 expression rates for subtypes
⁵Haematologica: http://www.haematologica.org/content/98/8/e81: CD30 expression by subtype

“Leukemia” as the term is used herein is a hematological malignancy that usually develops from hyper-proliferating cells of myeloid origin, and include without limitation, acute lymphoblastic leukemia (ALL), acute myelogenous leukemia (AML), chronic lymphocytic leukemia (CLL), chronic myelogenous leukemia (CML) and acute monocyctic leukemia (AMoL). Other leukemias include hairy cell leukemia (HCL), T-cell lymphatic leukemia (T-PLL), large granular lymphocytic leukemia and adult T-cell leukemia.

“Prophylactic” or “primary prophylaxis” as used herein refers to administration of an agent, such as a colony stimulating factor or granulopoiesis stimulating factor, prior to onset of neutropenia or symptoms of neutropenia in a subject. It is contemplated that prophylaxis includes administration of the granulopoeisis stimulating factor at initiation of, or first administration of, the anti-CD30-antibody drug conjugate therapy, or combination therapy comprising one or more chemotherapeutic agents. It is contemplated that a combination therapy comprises administering an anti-CD30 antibody drug conjugate and a chemotherapy consisting essentially of cyclophosphamide, doxorubicin and prednisone (CHP). The term “initiation” and “first administration” are used interchangeably herein in reference to treatment with granulopoiesis stimulating factor.

“Granulopoiesis stimulating factor” as used herein refers to an agent such as a cytokine or other growth factor that can induce production of neutrophils and other granulocytes. Exemplary granulopoiesis stimulating factors include, but are not limited to, granulocyte-colony stimulating factor (GCSF) and derivatives thereof, such as filgrastim and the long-acting GCSF PEG-filgrastim, or granulocyte-monocyte colony stimulating factor (GMCSF).

“Neutropenia” as used herein refers to an abnormally low concentration of neutrophils in the blood. “Reducing the incidence of neutropenia in a subject” refers to decreasing the number of neutropenia incidents in a subject receiving treatment and/or reducing the severity of neutropenic incidents in a subject. “Preventing neutropenia” refers to preventing or inhibiting the onset of neutropenia, e.g., as a result of prophylactic treatment with a granulopoiesis stimulating factor. Normal reference range for absolute neutrophil count (ANC) in adults is 1500 to 8000 cells per microliter (μl) of blood. Neutropenia can be categorized as follows: mild neutropenia (1000<=ANC<1500); moderate neutropenia (500<=ANC<1000); severe neutropenia (ANC<500). Hsieh et al., Ann. Intern. Med. 146:486-92, 2007.

The term “pharmaceutically acceptable” as used herein refers to those compounds, materials, compositions, and/or dosage forms that are, within the scope of sound medical judgment, suitable for contact with the tissues of human beings and animals without excessive toxicity, irritation, allergic response, or other problems or complications commensurate with a reasonable benefit/risk ratio. The term “pharmaceutically compatible ingredient” refers to a pharmaceutically acceptable diluent, adjuvant, excipient, or vehicle with which an antibody-drug conjugate is administered.

The terms “specific binding” and “specifically binds” mean that the anti-CD30 antibody will react, in a highly selective manner, with its corresponding target, CD30, and not with the multitude of other antigens.

The term “monoclonal antibody” refers to an antibody that is derived from a single cell clone, including any eukaryotic or prokaryotic cell clone, or a phage clone, and not the method by which it is produced. Thus, the term “monoclonal antibody” as used herein is not limited to antibodies produced through hybridoma technology.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=#of identical positions/total #of positions (e.g., overlapping positions)×100). In certain embodiments, the two sequences are the same length.

The term “substantially identical,” in the context of two nucleic acids or polypeptides, refers to two or more sequences or subsequences that have at least 70% or at least 75% identity; more typically at least 80% or at least 85% identity; and even more typically at least 90%, at least 95%, or at least 98% identity (for example, as determined using one of the methods set forth below).

The determination of percent identity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid encoding a protein of interest. BLAST protein searches can be performed with the XBLAST program, score=50, wordlength=3 to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. 85:2444-8. Alternatively, protein sequence alignment may be carried out using the CLUSTAL W algorithm, as described by Higgins et al., 1996, Methods Enzymol. 266:383-402.

The abbreviation “MMAE” refers to monomethyl auristatin E.

The abbreviations “vc” and “val-cit” refer to the dipeptide valine-citrulline.

The abbreviation “PAB” refers to the self-immolative spacer:

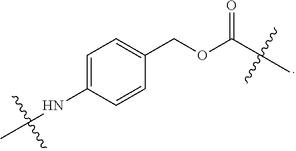 [SMILES: *Nc1ccc(COC(*)=O)cc1]

The abbreviation “MC” refers to the stretcher maleimidocaproyl:

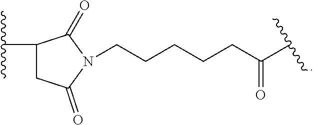 [SMILES: *C(=O)CCCCCN1C(=O)CC(*)C1=O]

cAC10-MC-vc-PAB-MMAE refers to a chimeric AC10 antibody conjugated to the drug MMAE through a MC-vc-PAB linker.

An anti-CD30 vc-PAB-MMAE antibody-drug conjugate refers to an anti-CD30 antibody conjugated to the drug MMAE via a linker comprising the dipeptide valine citrulline and the self-immolative spacer PAB as shown in Formula (I) of U.S. Pat. No. 9,211,319.

### Antibodies

Murine anti-CD30 mAbs known in the art have been generated by immunization of mice with Hodgkin's disease (HD) cell lines or purified CD30 antigen. AC10, originally termed C10 (Bowen et al., 1993, J. Immunol. 151:5896 5906), is distinct in that this anti-CD30 mAb that was prepared against a hum an NK-like cell line, YT (Bowen et al., 1993, J. Immunol. 151:5896 5906). Initially, the signaling activity of this mAb was evidenced by the down regulation of the cell surface expression of CD28 and CD45 molecules, the up regulation of cell surface CD25 expression and the induction of homotypic adhesion following binding of C10 to YT cells. Sequences of the AC10 antibody are set out in SEQ ID NO: 1-16 and Table A below. See also U.S. Pat. No. 7,090,843, incorporated herein by reference, which discloses a chimeric AC10 antibody.

Generally, antibodies of the disclosure immunospecifically bind CD30 and exert cytostatic and cytotoxic effects on malignant cells in Hodgkin's disease and mature T cell lymphoma. Antibodies of the disclosure are preferably monoclonal, and may be multispecific, human, humanized or chimeric antibodies, single chain antibodies, Fab fragments, F(ab) fragments, fragments produced by a Fab expression library, and CD30 binding fragments of any of the above. The term “antibody,” as used herein, refers to immunoglobulin molecules and immunologically active portions of immunoglobulin molecules, i.e., molecules that contain an antigen binding site that immunospecifically binds CD30. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the antibodies are human antigen-binding antibody fragments of the present disclosure and include, but are not limited to, Fab, Fab and F(ab), Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, may comprise the variable region(s) alone or in combination with the entirety or a portion of the following: hinge region, CH1, CH2, CH3 and CL domains. Also included in the disclosure are antigen-binding fragments also comprising any combination of variable region(s) with a hinge region, CH1, CH2, CH3 and CL domains. Preferably, the antibodies are human, murine (e.g., mouse and rat), donkey, sheep, rabbit, goat, guinea pig, camelid, horse, or chicken. As used herein, “human” antibodies include antibodies having the amino acid sequence of a human immunoglobulin and include antibodies isolated from human immunoglobulin libraries, from human B cells, or from animals transgenic for one or more human immunoglobulin, as described infra and, for example in U.S. Pat. No. 5,939,598 by Kucherlapati et al.

The antibodies of the present disclosure may be monospecific, bispecific, trispecific or of greater multi specificity. Multispecific antibodies may be specific for different epitopes of CD30 or may be specific for both CD30 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Antibodies of the present disclosure may be described or specified in terms of the particular CDRs they comprise. In certain embodiments antibodies of the disclosure comprise one or more CDRs of AC10. The disclosure encompasses an antibody or derivative thereof comprising a heavy or light chain variable domain, said variable domain comprising (a) a set of three CDRs, in which said set of CDRs are from monoclonal antibody AC10, and (b) a set of four framework regions, in which said set of framework regions differs from the set of framework regions in monoclonal antibody AC 10, and in which said antibody or derivative thereof immunospecifically binds CD30.

In a specific embodiment, the disclosure encompasses an antibody or derivative thereof comprising a heavy chain variable domain, said variable domain comprising (a) a set of three CDRs, in which said set of CDRs comprises SEQ ID NO:4, 6, or 8 and (b) a set of four framework regions, in which said set of framework regions differs from the set of framework regions in monoclonal antibody AC10, and in which said antibody or derivative thereof immunospecifically binds CD30.

In various embodiments, the disclosure encompasses an antibody or derivative thereof comprising a light chain variable domain, said variable domain comprising (a) a set of three CDRs, in which said set of CDRs comprises SEQ ID NO:12, 14 or 16, and (b) a set of four framework regions, in which said set of framework regions differs from the set of framework regions in monoclonal antibody AC10, and in which said antibody or derivative thereof immunospecifically binds CD30.

Additionally, antibodies of the present disclosure may also be described or specified in terms of their primary structures. Antibodies having at least 50%, at least 55%, at least 60%, at least 65%, at least 70%, at least 75%, at least 80%, at least 85%, at least 90%, at least 95% and most preferably at least 98% identity (as calculated using methods known in the art and described herein) to the variable regions of AC10 are also included in the present disclosure, and preferably include the CDRs of AC10. Antibodies of the present disclosure may also be described or specified in terms of their binding affinity to CD30. Preferred binding affinities include those with a dissociation constant or Kd less than 5×10² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

The antibodies also include derivatives that are modified, i.e., by the covalent attachment of any type of molecule to the antibody such that covalent attachment does not prevent the antibody from binding to CD30 or from exerting a cytostatic or cytotoxic effect on Hodgkin's Disease cells. For example, but not by way of limitation, the antibody derivatives include antibodies that have been modified, e.g., by glycosylation, acetylation, PEGylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present disclosure may be generated by any suitable method known in the art.

The disclosure further provides nucleic acids comprising a nucleotide sequence encoding a protein, including but not limited to, a protein of the disclosure and fragments thereof. Nucleic acids of the disclosure preferably encode one or more CDRs of antibodies that bind to CD30 and exert cytotoxic or cytostatic effects on HD cells. Exemplary nucleic acids of the disclosure comprise SEQ ID NO:3, SEQ ID NO:5, SEQ ID NO:7, SEQ ID NO:11, SEQ ID NO:13, or SEQ ID NO:15. Variable region nucleic acids of the disclosure comprise SEQ ID NO:1 or SEQ ID NO:9. (See Table A).

TABLE A

| | NUCLEOTIDE OR | SEQ |
|---|---|---|
| MOLECULE | AMINO ACID | ID NO |
| AC 10 Heavy Chain Variable Region | Nucleotide | 1 |
| AC 10 Heavy Chain Variable Region | Amino Acid | 2 |
| AC 10 Heavy Chain-CDR1 (H1) | Nucleotide | 3 |
| AC 10 Heavy Chain-CDR1 (H1) | Amino Acid | 4 |
| AC 10 Heavy Chain-CDR2 (H2) | Nucleotide | 5 |
| AC 10 Heavy Chain-CDR2 (H2) | Amino Acid | 6 |
| AC 10 Heavy Chain-CDR3 (H3) | Nucleotide | 7 |
| AC 10 Heavy Chain-CDR3 (H3) | Amino Acid | 8 |
| AC 10 Light Chain Variable Region | Nucleotide | 9 |
| AC 10 Light Chain Variable Region | Amino Acid | 10 |
| AC 10 Light Chain-CDR1 (L1) | Nucleotide | 11 |
| AC 10 Light Chain-CDR1 (L1) | Amino Acid | 12 |
| AC 10 Light Chain-CDR2 (L2) | Nucleotide | 13 |
| AC 10 Light Chain-CDR2 (L2) | Amino Acid | 14 |
| AC 10 Light Chain-CDR3 (L3) | Nucleotide | 15 |
| AC 10 Light Chain-CDR3 (L3) | Amino Acid | 16 |

In various embodiments, the antibody is an IgG antibody, e.g., an IgG1, IgG2, IgG3 or IgG4 antibody, preferably an IgG1 antibody.

### Antibody-Drug Conjugates

Contemplated herein is the use of antibody drug conjugates comprising an anti-CD30 antibody, covalently linked to MMAE through a vc-PAB linker. The antibody drug conjugates are delivered to the subject as a pharmaceutical composition. CD30 antibody drug conjugates are described in U.S. Pat. No. 9,211,319, herein incorporated by reference.

In various embodiments, the antibody-drug conjugates of the present disclosure have the following formula:

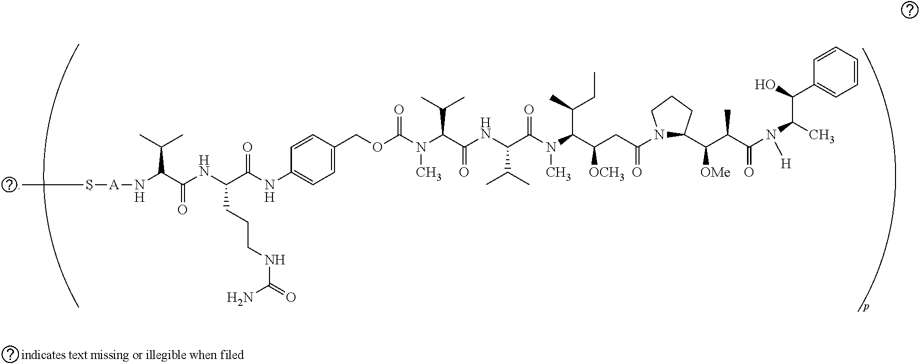

or a pharmaceutically acceptable salt thereof; wherein: mAb is an anti-CD30 antibody, S is a sulfur atom of the antibody A− is a Stretcher unit, p is from about 3 to about 5.

The drug loading is represented by p, the average number of drug molecules per antibody in a pharmaceutical composition. For example, if p is about 4, the average drug loading taking into account all of the antibody present in the pharmaceutical composition is about 4. P ranges from about 3 to about 5, more preferably from about 3.6 to about 4.4, even more preferably from about 3.8 to about 4.2. P can be about 3, about 4, or about 5. The average number of drugs per antibody in preparation of conjugation reactions may be characterized by conventional means such as mass spectroscopy, ELISA assay, and HPLC. The quantitative distribution of antibody-drug conjugates in terms of p may also be determined. In some instances, separation, purification, and characterization of homogeneous antibody-drug-conjugates where p is a certain value from antibody-drug-conjugates with other drug loadings may be achieved by means such as reverse phase HPLC or electrophoresis.

The Stretcher unit (A), is capable of linking an antibody unit to the valine-citrulline amino acid unit via a sulfhydryl group of the antibody. Sulfhydryl groups can be generated, for example, by reduction of the interchain disulfide bonds of an anti-CD30 antibody. For example, the Stretcher unit can be linked to the antibody via the sulfur atoms generated from reduction of the interchain disulfide bonds of the antibody. In some embodiments, the Stretcher units are linked to the antibody solely via the sulfur atoms generated from reduction of the interchain disulfide bonds of the antibody. In some embodiments, sulfhydryl groups can be generated by reaction of an amino group of a lysine moiety of an anti-CD30 antibody with 2-iminothiolane (Traut® reagent) or other sulfhydryl generating reagents. In certain embodiments, the anti-CD30 antibody is a recombinant antibody and is engineered to carry one or more lysines. In certain other embodiments, the recombinant anti-CD30 antibody is engineered to carry additional sulfhydryl groups, e.g., additional cysteines.

The synthesis and structure of MMAE is described in U.S. Pat. No. 6,884,869 incorporated by reference herein in its entirety and for all purposes. The synthesis and structure of exemplary Stretcher units and methods for making antibody drug conjugates are described in, for example, U.S. Publication Nos. 2006/0074008 and 2009/0010945 each of which is incorporated herein by reference in its entirety.

Representative Stretcher units are described within the square brackets of Formulas IIIa and IIIb of U.S. Pat. No. 9,211,319, and incorporated herein by reference.

In various embodiments, the antibody drug conjugate comprises monomethyl auristatin E and a protease-cleavable linker. It is contemplated that the protease cleavable linker is comprises a thiolreactive spacer and a dipeptide. In various embodiments, the protease cleavable linker consists of a thiolreactive maleimidocaproyl spacer, a valine-citrulline dipeptide, and a p-amino-benzyloxycarbonyl spacer.

In a preferred embodiment, the antibody drug conjugate is brentuximab vedotin, an antibody-drug conjugate which has the structure:

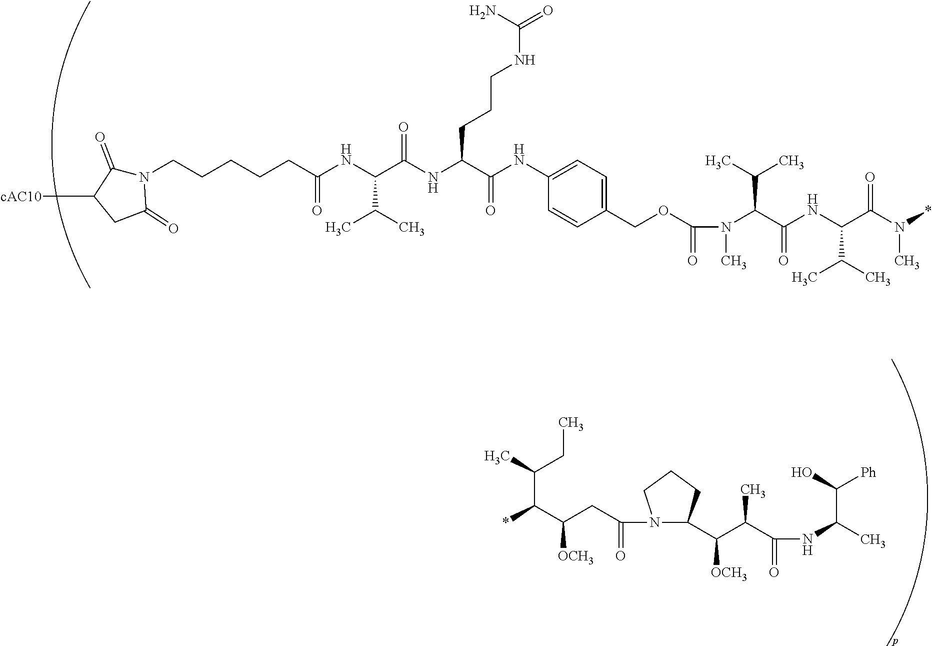

Brentuximab vedotin is a CD30-directed antibody-drug conjugate consisting of three components: (i) the chimeric IgG1 antibody cAC10, specific for human CD30, (ii) the microtubule disrupting agent MMAE, and (iii) a protease-cleavable linker that covalently attaches MMAE to cAC10. The drug to antibody ratio or drug loading is represented by “p” in the structure of brentuximab vedotin and ranges in integer values from 1 to 8. The average drug loading brentuximab vedotin in a pharmaceutical composition is about 4.

### Methods of Use

Provided herein are improved methods for administering anti-CD30 antibody-drug conjugate to a subject suffering from a mature T cell lymphoma. Disclosed herein are methods for reducing adverse events in a subject having a mature T cell lymphoma during administration of an anti-CD30 antibody drug conjugate, optionally in combination with a chemotherapy regimen. In various embodiments, the chemotherapy regimen consists essentially of cyclophosphamide, doxorubicin and/or prednisone, preferably as A+CHP therapy.

Additional chemotherapeutic agents are disclosed in the following table and may be used alone or in combination with one or more additional chemotherapeutic agents, which in turn can also be administered in combination with an anti-CD30 antibody drug conjugate.

### Chemotherapeutic Agents

| Alkylating agents | Natural products |
|---|---|
| Nitrogen mustards | Antimitotic drugs |
| mechlorethamine | Taxanes |
| cyclophosphamide | paclitaxel |
| ifosfamide | Vinca alkaloids |
| melphalan | vinblastine (VLB) |
| chlorambucil | vincristine |
| Nitrosoureas | vindesine |
| carmustine (BCNU) | vinorelbin |
| lomustine (CCNU) | Taxotere ® (docetaxel) |
| semustine (methyl-CCNU) | estramustine |
| Ethylenimine/Methyl-melamine | estramustine phosphate |
| thriethylenemelamine (TEM) | Epipodophylotoxins |
| triethylene thiophosphoramide | etoposide |
| (thiotepa) | teniposide |
| hexamethylmelamine | Antibiotics |
| (HMM, altretamine) | actimomycin D |
| Alkyl sulfonates | daunomycin (rubido-mycin) |
| busulfan | doxorubicin (adria-mycin) |
| Triazines | mitoxantrone |
| dacarbazine (DTIC) | idarubicin |
| Antimetabolites | epirubicin |
| Folic Acid analogs | valrubicin |
| methotrexate | bleomycin |
| Trimetrexate | splicamycin (mithramycin) |
| Pemetrexed | mitomycinC |
| (Multi-targeted antifolate) | dactinomycin |
| Pyrimidine analogs | aphidicolin |
| 5-fluorouracil | Enzymes |
| fluorodeoxyuridine | L-asparaginase |
| gemcitabine | L-arginase |
| cytosine arabinoside | Radiosensitizers |
| (AraC, cytarabine) | metronidazole |
| 5-azacytidine | misonidazole |
| 2,2 [symbol] difluorodeoxy-cytidine | desmethylmisonidazole |
| Purine analogs | pimonidazole |
| 6-mercaptopurine | etanidazole |
| 6-thioguanine | nimorazole |
| azathioprine | RSU 1069 |
| 2′-deoxycoformycin | EO9 |
| (pentostatin) | RB 6145 |
| erythrohydroxynonyl- | SR4233 |
| adenine (EHNA) | nicotinamide |
| fludarabine phosphate | 5-bromodeozyuridine |
| 2-chlorodeoxyadenosine | 5-iododeoxyuridine |
| (cladribine, 2-CdA) | bromodeoxycytidine |
| Type I Topoisomerase Inhibitors | Miscellaneous agents |
| camptothecin | bisphosphonates |
| topotecan | RANKL inhibitor |
| irinotecan | denosumab |
| Biological response modifiers | Platinium coordination complexes |
| G-CSF | cisplatin |
| GM-CSF | carboplatin |
| Differentiation Agents | oxaliplatin |
| retinoic acid derivatives | nthracenedione |
| Hormones and antagonists | mitoxantrone |
| Adrenocorticosteroids/antagonists | Substituted urea |
| calcitonin | hydroxyurea |
| prednisone and equiv-alents | Methylhydrazine derivatives |
| dexamethasone | N-methylhydrazine (MIH) |
| ainoglutethimide | procarbazine |
| Progestins | Adrenocortical suppressant |
| hydroxyprogesterone caproate | mitotane (o,p [symbol] DDD) |
| medroxyprogesterone acetate | ainoglutethimide |
| megestrol acetate | Cytokines |
| Estrogens | interferon (α, β, γ) |
| diethylstilbestrol | interleukin-2 |
| ethynyl estradiol/equivalents | Photosensitizers |
| Antiestrogen | hematoporphyrin derivatives |
| tamoxifen | Photofrin ® |
| Androgens | benzoporphyrin derivatives |
| testosterone propionate | Npe6 |
| fluoxymesterone/equivalents | tin etioporphyrin (SnET2) |
| Antiandrogens | pheoboride-a |
| flutamide | bacteriochlorophyll-a |
| gonadotropin-releasing | naphthalocyanines |
| hormone analogs | phthalocyanines |
| leuprolide | zinc phthalocyanines |
| Nonsteroidal antiandrogens | Radiation |
| flutamide | X-ray |
| Histone Deacetylase Inhibitors | ultraviolet light |
| Vorinostat | gamma radiation |
| Romidepsin | visible light |
| | infrared radiation |
| | microwave radiation |

A mature T cell lymphoma (MTCL) refers to a hematologic cancer that expresses the CD30 antigen. The CD30 antigen is expressed in large numbers on tumor cells of select lymphomas and leukemias, including, peripheral T cell lymphoma (PTCL), PTCL entities typically manifesting as nodal involvement, angioimmunoblastic T-cell lymphoma, anaplastic large cell lymphomas, peripheral T-cell lymphoma-not otherwise specified, subcutaneous panniculitis-like T-cell lymphoma, ALK-positive sALCL with an IPI score greater than or equal to 2, ALK-negative sALCL, PTCL-NOS, AITL, adult T-cell leukemia/lymphoma (ATLL; acute and lymphoma types only, must be positive for human T-cell leukemia virus 1), hepatosplenic gamma delta T-cell lymphoma, enteropathy-type intestinal T-cell lymphoma, and extranodal T-cell lymphoma-nasal type.

In any of the aspects or embodiments herein, the methods herein provide for treating a subject who is newly diagnosed and has not previously been treated for a mature T cell lymphoma, or a subject who has relapsed.

In various embodiments herein, the methods herein provide for treating a subject who is newly diagnosed and/or has not previously been treated for a peripheral T cell lymphoma, or a subject who has previously been treated for a peripheral T cell lymphoma, but has relapsed or the PTCL is refractory. In various embodiments, the peripheral T cell lymphoma is systemic anaplastic large cell lymphoma (sALCL), angioimmunoblastic T-cell lymphoma (AITL), peripheral T-cell lymphoma-not otherwise specified (PTCL-NOS), Adult T-Cell Leukemia/Lymphoma (ATLL), Enteropathy-associated T-cell lymphoma (EATL) and Hepatosplenic T-cell lymphoma.

In various embodiments, the disclosure provides a method of treating a subject having newly diagnosed mature T cell lymphoma comprising administering an effective amount of a combination therapy comprising brentuximab vedotin in combination with a chemotherapy consisting essentially of cyclophosphamide, doxorubicin, and prednisone (CHP therapy), wherein the brentuximab vedotin is administered at 1.8 mg/kg, cyclophosphamide is administered at 750 mg/m², doxorubicin is administered at 50 mg/m², and prednisone is administered at 100 mg on days 1 to 5 of a 21 day cycle. It is contemplated that the methods herein provide progression free survival (PFS) of the subject after therapy is maintained for greater than 6 months or 1 year. In various embodiments, the progression free survival (PFS) of the subject after therapy is maintained for approximately 2 years. In certain embodiments, after six to eight cycles of A+CHP therapy the subject has a Deauville score of 3 or less, or 2 or less.

### Peripheral Neuropathy

Peripheral neuropathy develops as a result of damage to the peripheral nervous system during treatment with anti-CD30 antibody drug conjugate. Symptoms include numbness or tingling, pricking sensations (paresthesia), and muscle weakness. Motor nerve damage is most commonly associated with muscle weakness.

Provided herein is a method for treating a subject having a mature T cell lymphoma that has exhibited Grade 2 or greater peripheral neuropathy after starting administration of a therapy comprising administration of an anti-CD30 antibody drug conjugate, e.g. brentuximab vedotin, at a dose of 1.8 mg/kg or more, comprising administering the anti-CD30 antibody drug conjugate at a dose of 0.9 to 1.2 mg/kg. In various embodiments, when the subject exhibits Grade 3 neuropathy, the administration of the anti-CD30 antibody drug conjugate, e.g., brentuximab vedotin, is withheld until peripheral neuropathy decreases to Grade 2 or lower and then 0.9 mg/kg to 1.2 mg/kg of the anti-CD30 antibody drug conjugate is administered. In various embodiments, the therapy further comprises a chemotherapy consisting essentially of cyclophosphamide (C), doxorubicin (H) and prednisone (P) as a combination therapy.

In various embodiments, when the subject exhibits Grade 3 neuropathy, the administration of anti-CD30 antibody drug conjugate is reduced, optionally to 0.9 to 1.2 mg/kg, until peripheral neuropathy decreases to Grade 2 or less and then 0.9 mg/kg to 1.2 mg/kg anti-CD30 antibody drug conjugate is administered or maintained. In some embodiments, the reduced dose of 0.9 to 1.2 mg/kg is given up to a maximum dose of 90 to 120 mg every 2 weeks or 3 weeks.

In certain embodiments, the subject exhibited Grade 2 or 3 peripheral neuropathy after starting anti-CD30 antibody drug conjugate administration at a dose of 1.8 mg/kg, optionally in combination with a chemotherapy consisting essentially of cyclophosphamide (C), doxorubicin (H) and prednisone (P) as a combination therapy.

In certain embodiments, the dose of anti-CD30 antibody drug conjugate is increased to 1.8 mg/kg or 1.2 mg/kg after the Grade 2 or Grade 3 peripheral neuropathy improves to Grade 1 or less, the administration optionally is in combination with a chemotherapy consisting essentially of cyclophosphamide (C), doxorubicin (H) and prednisone (P) as a combination therapy. In various embodiments, when the peripheral neuropathy is a Grade 2 or less or Grade 1 or less, treatment with anti-CD30 antibody drug conjugate is restarted at 1.2 mg/kg every two weeks, up to a maximum of 120 mg every 2 weeks.

Methods for measuring neuropathy are known in the art and utilized by the treating physician to monitor and diagnose neuropathy in a subject receiving anti-CD30 antibody drug conjugate therapy. For example, the National Cancer Information Center-Common Toxicity Criteria (NCIC-CCT) describes Grade 1 PN as characterized by mild paresthesias and/or loss of deep tendon flexion; Grade 2 PN is characterized by mile or moderate objective sensory loss and/or moderate paresthesias; Grade 3 PN is characterized by sensory loss and/or paresthesias that interferes with function. Grade 4 PN is characterized by paralysis.

In various embodiments, if the anti-CD30 antibody drug conjugate is administered at 1.8 mg/kg with CHP combination therapy, the combination therapy is administered every three weeks. For example, the combination therapy is administered on day 1 of a 21 day cycle.

In various embodiments, the antibody drug conjugate+CHP combination therapy is administered for no more than eight cycles, for example from 4 to 8 cycles, or for 4, 5, 6, 7 or 8 cycles. Optionally, single agent therapy may be given after completion of combination therapy, for 8 to 10 cycles, or additional cycles as appropriate, for a total of 16 cycles.

It is contemplated that the combination therapy may also include administration of vincristine (e.g., Oncovin).

It is contemplated that the therapy, e.g. ADC or combination therapy, is administered until a PET scan determines there is no tumor or progression of tumor. If after the end of treatment, e.g., 6 to 8 cycles, the PET scan still shows some tumor, the treating physician may repeat the course of treatment as necessary until the PET scan is negative or shows slowed or no tumor progression. The repeat of cycles may begin after no break, or after 1, 2, 3, 4, 5, 6 or more weeks after the initial treatment with the therapy.

In various embodiments, anti-CD30 antibody drug conjugate, e.g., brentuximab vedotin, is administered by intravenous infusion over the course of 30 minutes. In certain embodiments, the anti-CD30 antibody drug conjugate is administered at 1.8 mg/kg to a maximum of 180 mg in combination every three weeks with CHP therapy.

The treatment is useful to treat peripheral motor neuropathy or peripheral sensory neuropathy. The treatment reduces one or more symptoms of peripheral neuropathy, including but not limited to, paresthesia, hypoesthesia, polyneuropathy, muscular weakness, and demyelinating polyneuropathy.

In various embodiments, the dose of anti-CD30 antibody drug conjugate is delayed by one week, or two weeks, if peripheral neuropathy appears, and therapy is continued when the neuropathy is resolved or determined to be Grade 2 or less or Grade 1 or less.

### Neutropenia

Neutropenia is a common side effect of chemotherapy regimens and results from depletion of neutrophils in the blood of patients receiving chemotherapeutic treatment. Neutropenia is also observed in treatment with brentuximab vedotin. Neutropenia is commonly diagnosed based on levels of neutrophils in the blood. For example, Grade 3 neutropenia refers to an absolute blood neutrophil count [ANC]<1.0×10⁹/l); Grade 4 neutropenia refers to absolute blood neutrophil count [ANC]<0.5×10⁹/l), Febrile neutropenia refers to neutropenia with fever, the subject having a single oral temperature ≥38.3° C. or ≥38.0° C. for ≥1 h, with grade 3/4 neutropenia.

It is contemplated herein that subjects receiving an anti-CD30 antibody drug conjugate, e.g., brentuximab vedotin, or anti-CD30 antibody drug conjugate in combination with chemotherapy, such as CHP combination therapy, receive granulopoiesis stimulating factors prophylactically, e.g., as a primary prophylaxis at initiation of, or first administration of, the therapy, e.g., ADC or combination therapy. Exemplary granulopoiesis stimulating factors include granulocyte colony stimulating factor (GCSF), derivatives of GCSF, or granulocyte monocyte colony stimulating factor (GMCSF). Commercially available GCSF contemplated for use herein are filgrastim (NEUPOGEN®) and pegfilgrastim (NEULASTA®). Commercially available GMCSF is available as sargramostim (LEUKINE®).

Provided herein is a method for treating a mature T cell lymphoma in a subject comprising administering a therapy comprising an anti-CD30 antibody drug conjugate, optionally the therapy further comprises a chemotherapy consisting essentially of cyclophosphamide (C), doxorubicin (H) and prednisone (P) as a combination therapy and prophylactically administering a granulopoiesis stimulating factor, wherein the granulopoiesis stimulating factor is administered within 1 day to within 7 days after the initiation of the therapy, e.g., ADC or combination therapy. In further embodiments, the granulopoiesis stimulating factor is administered from within 1 day to within 5 days after the initiation of the therapy, e.g., antibody drug conjugate or combination therapy. In some embodiments, the method is a method for decreasing adverse events associated with anti-CD30 antibody drug conjugate administration, e.g. neutropenia, febrile neutropenia, incidence of infection, pyrexia, gastrointestinal disorders such as constipation, vomiting, diarrhea, stomatitis, abdominal pain, nervous system disorders such as peripheral sensory neuropathy, peripheral motor neuropathy, musculoskeletal disorders such as bone pain, back pain, respiratory disorders such as dyspnea, and other adverse events such as decreased weight, increased alanine aminotransferase, decreased appetite and/or insomnia. In some embodiments, the method is a method for decreasing neutropenia and/or febrile neutropenia and/or incidence of infection associated with anti-CD30 antibody drug conjugate administration.

Also provided is a method for decreasing the incidence of infection in a subject receiving a therapy comprising an anti-CD30 antibody drug conjugate, optionally further comprising a chemotherapy consisting essentially of cyclophosphamide (C), doxorubicin (H) and prednisone (P), comprising administering to the subject granulopoiesis stimulating factor in an amount effective to reduce infections, wherein the granulopoiesis stimulating factor is administered from 1 day to 7 days after the initiation of the therapy. The granulopoiesis stimulating factor may also be administered from 1 days to 5 days after the initiation of the therapy.

Also contemplated is a method for reducing the incidence of neutropenia and/or febrile neutropenia in a subject receiving a therapy comprising an anti-CD30 antibody drug conjugate, optionally also with combination therapy comprising anti-CD30 antibody drug conjugate with a chemotherapy consisting essentially of cyclophosphamide (C), doxorubicin (H) and prednisone (P), comprising administering to the subject a granulopoiesis stimulating factor, wherein the stimulating factor is administered from 1 day to 7 days after initiation of the of the therapy, optionally from 1 days to 5 days after the initiation of the therapy.

Further contemplated is a method wherein the granulopoiesis stimulating factor is administered from 1 day to 7 days after a second, or subsequent, administration of the therapy, e.g., ADC or combination therapy. In certain embodiments, the granulopoiesis stimulating factor is administered from 1 day or 2 days to 5 days after the second or subsequent administration of the therapy.

In various embodiments, the subject has not received anti-CD30 antibody drug conjugate therapy previously. In various embodiments, the subject has not experienced treatment-emergent Grade 3-4 neutropenia after anti-CD30 antibody drug conjugate administration.

It is contemplated that the granulopoiesis stimulating factor is granulocyte colony stimulating factor (GCSF). It is contemplated that the GCSF is a long-acting GCSF or not a long acting GCSF.

In various embodiments, when the stimulating factor is not long-acting GCSF, e.g. filgrastim, it can be administered starting from 1 to 7 days, from 1 to 5 days, or 1 to 3 days after initiation of therapy, e.g. in daily doses. In certain embodiments, the GCSF is administered on day 2, 3, 4, 5, 6 and/or 7 after initiation of antibody drug conjugate or combination therapy. In various embodiments, the filgrastim is administered at a dose of 5 ug/kg/day to 10 ug/kg/day for the duration of at least 3, 4, 5, 6, 7, 8, 9, 10, 11, 12, 13 or 14 days.

Pegfilgrastim is a long-lasting, PEGylated form of filgrastim that has a longer half-life in vivo. In various embodiments, pegfilgrastim is administered at 6 mg/dose from 1 day to 5 days after anti-CD30 antibody drug conjugate treatment, or optionally after A+CHP therapy. In certain embodiments, the GCSF is administered in a single dose, or a multiple dose on the same day, on day 1, day 2, day 3, day 4 or day 5 after initiation of the therapy.

In various embodiments, when the lymphoma is peripheral T cell lymphoma, the granulopoiesis stimulating factor can be administered from 1 to 8 days after anti-CD30 antibody drug conjugate administration.

In various embodiments, the granulopoiesis stimulating factor is administered intravenously or subcutaneously. It is contemplated that the granulopoiesis stimulating factor is given in a single dose or multiple doses, e.g., in multiple daily doses.

It is contemplated that a subject receiving a granulopoiesis stimulating factor and anti-CD30 antibody drug conjugate or combination therapy may also be administered an antibiotic to address issues of febrile neutropenia and/or infection. Exemplary antibiotics contemplated include those known in the art, such as cephalosporin, sulfamethoxazole-trimethoprim, ACYCOLOVIR®, FLUCANOZOLE®, or INTRACONAZOLE®.

In various embodiments, the anti-CD30 antibody drug conjugate, or combination therapy, is administered every 3 weeks, e.g., on day 1 of a 21 day cycle. In various embodiments, when the anti-CD30 antibody drug conjugate is administered every 3 weeks, the regimen further comprises administering a chemotherapy consisting essentially of cyclophosphamide (C), doxorubicin (H) and prednisone (P) as a combination therapy, on the same day as the anti-CD30 antibody therapy.

In various embodiments, the anti-CD30 antibody drug conjugate is administered every 2 weeks when the dose administration has been reduced due to an adverse event.

In various embodiments, the mature T cell lymphoma is selected from the group consisting of peripheral T cell lymphoma (PTCL), PTCL entities typically manifesting as nodal involvement, angioimmunoblastic T-cell lymphoma, anaplastic large cell lymphomas, peripheral T-cell lymphoma-not otherwise specified, subcutaneous panniculitis-like T-cell lymphoma, hepatosplenic gamma delta T-cell lymphoma, enteropathy-type intestinal T-cell lymphoma, and extranodal T-cell lymphoma-nasal type.

It is further contemplated that upon completion of therapy with anti-CD30 antibody drug conjugate as described herein, optionally in combination with a chemotherapy regimen, the subject may receive an additional treatment to address one or more symptoms of cancer that remains at the end of treatment, or may be refractory to the therapy herein. Such treatments include, but are not limited to surgery, radiation therapy, proton beam therapy, stem cell transplant, and/or additional chemotherapeutic regimens.

### Formulations

Various delivery systems can be used to administer antibody-drug conjugates. In certain preferred embodiments of the present disclosure, administration of the antibody-drug conjugate compound is by intravenous infusion. In some embodiments, administration is by a 30 minute, 1 hour or two hour intravenous infusion.

The antibody-drug conjugate compound can be administered as a pharmaceutical composition comprising one or more pharmaceutically compatible ingredients. For example, the pharmaceutical composition typically includes one or more pharmaceutically acceptable carriers, for example, water-based carriers (e.g., sterile liquids). Water is a more typical carrier when the pharmaceutical composition is administered intravenously.

The composition, if desired, can also contain, for example, saline salts, buffers, salts, nonionic detergents, and/or sugars. Examples of suitable pharmaceutical carriers are described in “Remington® Pharmaceutical Sciences” by E. W. Martin. The formulations correspond to the mode of administration.

The present disclosure provides, for example, pharmaceutical compositions comprising a therapeutically effective amount of the antibody-drug conjugate, a buffering agent, optionally a cryoprotectant, optionally a bulking agent, optionally a salt, and optionally a surfactant. Additional agents can be added to the composition. A single agent can serve multiple functions. For example, a sugar, such as trehalose, can act as both a cryoprotectant and a bulking agent. Any suitable pharmaceutically acceptable buffering agents, surfactants, cyroprotectants and bulking agents can be used in accordance with the present disclosure.

In addition to providing methods for treating a CD30-expressing cancer, the present disclosure provides antibody drug conjugate formulations including drug conjugate formulations that have undergone lyophilization, or other methods of protein preservation, as well as antibody drug formulations that have not undergone lyophilization.

In some embodiments, the antibody drug conjugate formulation comprises (i) about 1-25 mg/ml, about 3 to about 10 mg/ml of an antibody-drug conjugate, or about 5 mg/ml (e.g., an antibody-drug conjugate of formula I or a pharmaceutically acceptable salt thereof), (ii) about 5-50 mM, preferably about 10 mM to about 25 mM of a buffer selected from a citrate, phosphate, or histidine buffer or combinations thereof, preferably sodium citrate, potassium phosphate, histidine, histidine hydrochloride, or combinations thereof, (iii) about 3% to about 10% sucrose or trehalose or combinations thereof, (iv) optionally about 0.05 to 2 mg/ml of a surfactant selected from polysorbate 20 or polysorbate 80 or combinations thereof; and (v) water, wherein the pH of the composition is from about 5.3 to about 7, preferably about 6.6.

In some embodiments, an antibody drug conjugate formulation will comprise about 1-25 mg/ml, about 3 to about 10 mg/ml, preferably about 5 mg/ml of an antibody-drug conjugate, (ii) about 10 mM to about 25 mM of a buffer selected from sodium citrate, potassium phosphate, histidine, histidine hydrochloride or combinations thereof, (iii) about 3% to about 7% trehalose or sucrose or combinations thereof, optionally (iv) about 0.05 to about 1 mg/ml of a surfactant selected from polysorbate 20 or polysorbate 80, and (v) water, wherein the pH of the composition is from about 5.3 to about 7, preferably about 6.6.

In some embodiments, an antibody drug conjugate formulation will comprise about 5 mg/ml of an antibody-drug conjugate, (ii) about 10 mM to about 25 mM of a buffer selected from sodium citrate, potassium phosphate, histidine, histidine hydrochloride or combinations thereof, (iii) about 3% to about 7% trehalose, optionally (iv) about 0.05 to about 1 mg/ml of a surfactant selected from polysorbate 20 or polysorbate 80, and (v) water, wherein the pH of the composition is from about 5.3 to about 7, preferably about 6.6.

Any of the formulations described above can be stored in a liquid or frozen form and can be optionally subjected to a preservation process. In some embodiments, the formulations described above are lyophilized, i.e., they are subjected to lyophilization. In some embodiments, the formulations described above are subjected to a preservation process, for example, lyophilization, and are subsequently reconstituted with a suitable liquid, for example, water. By lyophilized it is meant that the composition has been freeze-dried under a vacuum. Lyophilization typically is accomplished by freezing a particular formulation such that the solutes are separated from the solvent(s). The solvent is then removed by sublimation (i.e., primary drying) and next by desorption (i.e., secondary drying).

The formulations of the present disclosure can be used with the methods described herein or with other methods for treating disease. The antibody drug conjugate formulations may be further diluted before administration to a subject. In some embodiments, the formulations will be diluted with saline and held in IV bags or syringes before administration to a subject. Accordingly, in some embodiments, the methods for treating a mature T cell lymphoma in a subject will comprise administering to a subject in need thereof a weekly dose of a pharmaceutical composition comprising antibody-drug conjugates having formula I wherein the administered dose of antibody-drug conjugates is from about 1.8 mg/kg or 1.2 mg/kg of the subjects body weight to 0.9 mg/kg of the subjects body weight and the pharmaceutical composition is administered for at least three weeks and wherein the antibody drug conjugates, prior to administration to a subject, were present in a formulation comprising (i) about 1-25 mg/ml, preferably about 3 to about 10 mg/ml of the antibody-drug conjugate (ii) about 5-50 mM, preferably about 10 mM to about 25 mM of a buffer selected from sodium citrate, potassium phosphate, histidine, histidine hydrochloride, or combinations thereof, (iii) about 3% to about 10% sucrose or trehalose or combinations thereof, (iv) optionally about 0.05 to 2 mg/ml of a surfactant selected from polysorbate 20 or polysorbate 80 or combinations thereof; and (v) water, wherein the pH of the composition is from about 5.3 to about 7, preferably about 6.6.

Formulations of chemotherapeutics contemplated for use herein, including cyclophosphamide, doxorubicin and prednisone are provided as typically used in the treatment of cancers. For example, cyclophosphamide, doxorubicin, and prednisone are commercially available and approved by the United States FDA and other regulatory agencies for use in treating patients with multiple types of cancer. Vincristine is commercially available and approved by the United States FDA and other regulatory agencies for use in patients with multiple types of cancer.

Administration of study treatment should be according to the institutional standard. Dosing should be based on the patient's baseline (predose, Cycle 1 Day 1) height and weight or per institutional standards at the site. Vincristine is typically administered as an IV push, and will be given on Day 1 of each 21-day cycle. Dosing should be based on the patient's baseline (predose, Cycle 1 Day 1) height and weight or per institutional standards at the site.

The present disclosure also provides kits for the treatment of a mature T cell lymphoma. The kit can comprise (a) a container containing the antibody-drug conjugate and optionally, containers comprising one or more of cyclophosphamide, doxorubicin and/or prednisone. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

### Examples

The clinical safety and activity of brentuximab vedotin administered sequentially and concurrently with multiagent chemotherapy were previously evaluated in a phase 1 study in patients with newly diagnosed CD30-positive mature T- and NK-cell neoplasms, including sALCL (Study SGN35-011). This phase 1 study was implemented to determine the safety and activity of sequential and combination frontline treatment approaches of brentuximab vedotin with CHOP or CHP chemotherapy. The maximum tolerated dose of brentuximab vedotin was 1.8 mg/kg given concomitantly with CHP. At an interim analysis in this study (data presented at the T-Cell Lymphoma Forum 2012), 20 patients in this study had been treated with brentuximab vedotin 1.2 or 1.8 mg/kg given concomitantly with CHP for 6 cycles, followed by continued brentuximab vedotin every 3 weeks for up to 10 additional cycles for responding patients. The most common adverse events were nausea, fatigue, and peripheral sensory neuropathy. Of the patients who had a response assessment after 6 cycles of brentuximab vedotin plus CHP, 5 of 5 patients achieved a CR.

Given the results of treatment with brentuximab vedotin in the relapsed and refractory setting, and its demonstrated safety when combined with CHP in a Phase I study, it is hypothesized that a treatment approach in adults that incorporates brentuximab vedotin as part of multiagent frontline induction therapy may yield a progression free survival (PFS) and overall survival (OS) benefit. It is also reasonable to evaluate the replacement of vincristine with brentuximab vedotin because of the activity previously observed. By replacing a non-targeted microtubule-disrupting agent with a CD30-directed ADC that delivers a potent microtubule-disrupting agent, the potential overlapping toxicities of peripheral neuropathy that would be inherent to delivering both agents in the same regimen are avoided.

Described below is a randomized, double-blind, placebo-controlled, multicenter, Phase 3 clinical trial designed to evaluate the efficacy and safety of including brentuximab vedotin in the treatment of newly diagnosed, CD30-positive mature T-cell lymphomas as a frontline therapy.

The primary endpoint of this study, PFS, is one of the endpoints recommended by FDA (FDA Guidance for Industry “Clinical Trial Endpoints for the Approval of Cancer Drugs and Biologics”) and the EMA (“Guideline on the Evaluation of Anticancer Medicinal Products in Man”, CPMP/EWP/205/95/Rev.3/Corr.2) for approval of anticancer drugs. Defined as the time from randomization until objective tumor progression or death, PFS is a direct reflection of tumor growth and can be assessed before determination of a survival benefit. Furthermore, because PFS includes deaths from any cause it may be a correlate to overall survival, a secondary endpoint of this study. An additional advantage of PFS is that its determination is not confounded by subsequent therapy. In this study, post treatment radiotherapy, post treatment chemotherapy for the purpose of mobilizing peripheral blood stem cells, or consolidative autologous or allogeneic SCT are not considered subsequent new anticancer treatments because they are not administered to treat progressive disease.

Standardized criteria is employed to evaluate progression (Cheson 2007). To ensure consistent unbiased application of these criteria, all imaging studies performed to confirm disease status and to assess progression during the study will be submitted to an independent third-party imaging core laboratory for blinded review and all patients will have evaluations for progression performed on the same schedule.

### Materials and Methods

TRIAL DESIGN: Approximately 450 patients (approximately 225 patients per treatment arm) are randomized in this study. The standard of care in this patient population consists of 6-8 cycles of CHOP chemotherapy. Patients are randomized in a 1:1 manner to receive 21-day cycles of treatment in 1 of the following 2 treatment groups: Standard-of-care arm: 6-8 cycles of CHOP; or Experimental arm: 6-8 cycles of brentuximab vedotin plus CHP (A+CHP). A target of 8 cycles of study treatment are administered, per investigator decision, based on patient-specific characteristics, including stage of disease and IPI risk score.

PATIENTS: Patients with newly diagnosed, CD30-positive mature T-cell lymphomas per the Revised European-American Lymphoma WHO 2008 classification by local assessment are included in the study. Eligible histologies are limited to the following: ALK-positive sALCL with an IPI score greater than or equal to 2; ALK-negative sALCL; PTCL-NOS; AITL; Adult T-cell leukemia/lymphoma (ATLL; acute and lymphoma types only, must be positive for human T-cell leukemia virus 1); Enteropathy-associated T-cell lymphoma (EATL); Hepatosplenic T-cell lymphoma; Fluorodeoxyglucose (FDG)-avid disease by PET and measurable disease of at least 1.5 cm by CT, as assessed by the site radiologist, and age greater than or equal to 18 years. Patients were required to have an Eastern Cooperative Oncology Group performance status ≥2, and satisfactory absolute neutrophil and platelet counts, hemoglobin levels, and liver and kidney function marker levels.

Exclusion criteria includes history of another primary invasive cancer, hematologic malignancy, or myelodysplastic syndrome that has not been in remission for at least 3 years. No subjects should have current diagnosis of any of the following: Primary cutaneous CD30-positive T-cell lymphoproliferative disorders and lymphomas. Cutaneous ALCL with extracutaneous tumor spread beyond locoregional lymph nodes is eligible (previous single-agent treatment to address cutaneous and locoregional disease is permissible), Mycosis fungoides (MF), including transformed MF, History of progressive multifocal leukoencephalopathy (PML), Cerebral/meningeal disease related to the underlying malignancy, Prior treatment with brentuximab vedotin, Baseline peripheral neuropathy Grade 2 (per the NCI CTCAE, Version 4.03) or patients with the demyelinating form of Charcot-Marie-Tooth syndrome.

Optionally, granulopoiesis stimulating factor is administered prophylactically if the patient experiences neutropenia. The granulopoiesis stimulating factor is administered using standard regimens known in the art. It is contemplated that administration of granulopoiesis stimulating factor at the initiation of treatment will reduce the incidence of neutropenia and/or infection.

ENDPOINTS: The primary endpoint is modified progression-free survival (PFS), defined as time to progression, death, or evidence of non-CR after completion of frontline therapy per independent review facility (IRF). Timing of the modified event is the date of the first PET scan post-completion of frontline therapy demonstrating the absence of CR, defined as Deauville score of ≥3. In the absence of disease progression a switch to an alternative frontline therapy, for any reason, prior to completion of treatment with the randomized regimen was not considered an event.

Secondary endpoints include PFS per IRF for patients with sALCL, Complete remission (CR) rate per IRF following the completion of study treatment, Overall survival (OS) defined as time from randomization to death due to any cause, Objective response rate (ORR) per IRF following the completion of study treatment, Type, incidence, severity, seriousness, and relatedness of adverse events. Complete remission (CR) rate is defined as the proportion of patients with CR at the end of treatment per IRF according to the Revised Response Criteria for Malignant Lymphoma (Cheson 2007). Patients whose disease response cannot be assessed will be scored as nonresponders for calculating the CR rate.

Overall survival (OS) is defined as the time from randomization to death due to any cause. Specifically, OS=Date of death−Date of randomization+1. For a patient who is not known to have died by the end of study follow-up, observation of OS is censored on the date the patient was last known to be alive (i.e., date of last contact). Patients lacking data beyond the day of randomization will have their survival time censored on the date of randomization (i.e., OS duration of 1 day). ORR per IRF is defined as the proportion of patients with CR or partial remission (PR) per IRF following the completion of study treatment (at EOT) according to the Revised Response Criteria for Malignant Lymphoma (Cheson 2007).

Additional Endpoints include incidence of anti-therapeutic antibodies (ATA) to brentuximab vedotin (defined as the proportion of patients that develop ATA at any time during the study), Medical Resource Utilization based on the number of medical care encounters, Quality of life measured by the European Organisation for Research and Treatment of Cancer (EORTC) core quality of life questionnaire (QLQ-C30) and European Quality of Life 5-Dimensional (EQ-5D).

ASSESSMENTS: Response and progression are evaluated as set out above. Computed tomography scans are performed at screening, after Cycle 4, after the last dose of frontline therapy and, during the follow-up period, every 3 months for the first two years and 6 months thereafter. PET scans are conducted at screening, at the end of Cycle 4 and end of treatment.

Safety is evaluated by the incidence of adverse events, using the Medical Dictionary for Regulatory Activities (MedDRA; v19.0), and National Cancer Institute Common Terminology Criteria for Adverse Events v4.03, and by changes in vital signs, and clinical laboratory results.

Patient reported outcome questionnaires are performed periodically throughout treatment, e.g., during every cycle. The European Quality of Life (EuroQOL) EQ-5D is a 5-item questionnaire with a “thermometer” visual analog scale ranging from 0 (worst imaginable health state) to 100 (best imaginable health state).

The FACT/GOG-NTX is a self-administered questionnaire for assessing changes in quality of life and assessment of treatment-induced neurologic symptoms (sensory, hearing, motor, and dysfunction). Patients score their well-being by selecting the frequency with which they associate with a given statement (0 being “not at all”, up to 4 being “very much”). The neurotoxicity subscale consists of 11 questions.

The EORTC QLQ-C30 is a questionnaire developed to assess the quality of life of cancer patients. The QLQ-C30 incorporates 9 multi-item scales: 5 functional scales (physical, role, cognitive, emotional, and social), 3 symptom scales (fatigue, pain, and nausea and vomiting), and a global health and quality of life scale (Aaronson 1993).

All efficacy evaluations are conducted using the intent-to-treat population unless otherwise specified. Safety is analyzed in patients who received at least one dose of study drug (safety population).

It is provided that treatment with A+CHP therapy reduces adverse effects such as peripheral neuropathy and hepatic or renal impairment when doses of brentuximab vedotin are reduced after appearance of Grade 2 or greater neuropathy in a subject. The doses may be reduced to 1.2 mg/kg or 0.9 mg/kg or a dose within that range.

Further, prophylactic administration of a granulopoiesis stimulating factor, such as GCSF, at the initiation of treatment with A+CHP therapy reduces the incidence of neutropenia in subjects, including febrile neutropenia. Rates of infection may also be decreased with prophylactic administration of a granulopoiesis stimulating factor.

### Results and Discussion

A total of 452 subjects were randomized on study: 226 to the A+CHP arm and 226 to the CHOP arm. A total of 370 subjects (82%) completed treatment; 192 subjects (85%) on the A+CHP arm and 178 subjects (79%) on the CHOP arm. As of the 15 Aug. 2018 data cutoff date, 296 subjects (65%) remained in long-term follow up; 157 subjects (69%) on the A+CHP arm and 139 subjects (62%) on the CHOP arm. The overall median age was 58 years (range, 18 to 85). Most subjects were male (63%) and white (62%). The protocol required 75%±5% of subjects to have a diagnosis of sALCL to support the secondary endpoint of PFS in this population; therefore, 316 of 452 enrolled subjects (70%) had a diagnosis of sALCL per local assessment. Of the 316 subjects with sALCL, 218 (69%) were ALK-negative (48% of the total population of randomized subjects). The median time from initial disease diagnosis to first dose of study treatment was 0.9 months (range, 0 to 19 months). Overall, 53% of subjects had Stage IV disease at initial diagnosis. There were no meaningful differences in demographics and baseline characteristics between the treatment arms.

Subjects were randomly assigned in a 1:1 ratio to receive 21-day cycles of either A+CHP or CHOP for 6 or 8 cycles, with the number of cycles determined at the outset and based on investigator discretion. Vincristine was omitted from combination treatment with brentuximab vedotin to eliminate the potential for additional neurotoxicity. All subjects were administered the CHP components of the CHOP regimen (cyclophosphamide 750 mg/m2 and doxorubicin 50 mg/m2 administered IV on Day 1 of each cycle; prednisone 100 mg daily administered orally on Days 1 to 5 of each cycle). Brentuximab vedotin (A+CHP arm; 1.8 mg/kg administered IV on Day 1 of each cycle) or vincristine (CHOP arm; 1.4 mg/m2 [maximum 2.0 mg] administered IV on Day 1 of each cycle) were dispensed after CHP to subjects in a double-blind, active-controlled manner (subjects received either brentuximab vedotin and a vincristine placebo or vincristine and a brentuximab vedotin placebo). Post treatment consolidative SCT or radiotherapy was permitted at the investigator's discretion after at least 6 cycles of study treatment were administered (intent was pre-specified).

Randomization was stratified by histologic subtype per local pathology assessment (ALK-positive sALCL vs. all other histologies) and baseline International Prognostic Index (IPI) score 16 (0-1 vs. 2-3 vs. 4-5).

The primary and all key secondary endpoints of this study were met and were statistically significant. The primary endpoint of this study, Progression-free survival (PFS) per independent review facility (IRF), was defined as the time from the date of randomization to the date of first documentation of progressive disease (PD), death due to any cause, or receipt of subsequent anticancer chemotherapy to treat residual or progressive disease, whichever occurred first. Receipt of post-treatment consolidative radiotherapy, post treatment chemotherapy for the purpose of mobilizing peripheral stem cells, or consolidative autologous or allogeneic SCT was not considered disease progression or as having started new anticancer therapy.

The study results show that PFS per IRF was significantly improved on the A+CHP arm compared with the CHOP arm (stratified HR 0.71 [95% CI: 0.54, 0.93], P=0.011). The difference equates to a 29% reduction in the risk of PFS events (disease progression, death, or receipt of new therapy) for A+CHP versus CHOP.

### Secondary Endpoint Analysis

There was a 41% reduction in risk of PFS events per IRF for the subset of subjects with sALCL on the A+CHP arm compared to the CHOP arm (HR 0.59 [95% CI: 0.42, 0.84], P=0.0031), consistent with the results of the primary analysis.

The complete response (CR) rate at end of treatment (EOT) by IRF assessment was 68% (95% CI: 61.2, 73.7) for subjects on the A+CHP arm compared with 56% (95% CI: 49.0, 62.3) for subjects on the CHOP arm. The CR rate difference between the arms was statistically significant by stratified Cochran-Mantel-Haenszel (CMH) test (P=0.0066). Overall survival (OS) was significantly improved with A+CHP versus CHOP (P=0.024). The stratified HR was 0.66 (95% CI: 0.46, 0.95), which equates to a 34% reduction in the risk of death for subjects treated with A+CHP versus CHOP. As of the time of the primary analysis, 124 subjects (27%) had died; 51 subjects (23%) on the A+CHP arm versus 73 subjects (32%) on the CHOP arm.

The overall response rate (ORR) at EOT by IRF assessment was 83% (95% CI: 77.7, 87.8) for subjects on the A+CHP arm compared with 72% (95% CI: 65.8, 77.9) for subjects on the CHOP arm. The response rate difference was statistically significant by stratified CMH test (P=0.0032).

The following Tables 1-6 show the detailed analysis on PFS per IRF and OS for various subgroups:

TABLE 1
Analysis on PFS per IRF and OS based on IPI Scores

| | PFS per IRF Subgroup Analysis | | | Overall Survival Subgroup Analysis | | |
|---|---|---|---|---|---|---|
| | Event/N | | Hazard Ratio | Event/N | | Hazard Ratio |
| IPI Score | A + CHP | CHOP | (95% CI) | A + CHP | CHOP | (95% CI) |
| 0-1 | 18/52 | 27/48 | 0.53 | 5/52 | 10/48 | 0.46 |
| | | | (0.29, 0.97) | | | (0.16, 1.33) |
| 2-3 | 56/141 | 77/145 | 0.71 | 29/141 | 48/145 | 0.56 |
| | | | (0.50, 1.00) | | | (0.35, 0.89) |
| 4-5 | 21/33 | 20/33 | 1.03 | 17/33 | 15/33 | 1.15 |
| | | | (0.55, 1.92) | | | (0.58, 2.31) |

TABLE 2
Analysis on PFS per IRF and OS based on Age

| | PFS per IRF Subgroup Analysis | | | Overall Survival Subgroup Analysis | | |
|---|---|---|---|---|---|---|
| | Event/N | | Hazard Ratio | Event/N | | Hazard Ratio |
| Age | A + CHP | CHOP | (95% CI) | A + CHP | CHOP | (95% CI) |
| <65 years | 54/157 | 75/156 | 0.67 | 26/157 | 37/156 | 0.64 |
| | | | (0.47, 0.95) | | | (0.39, 1.06) |
| ≥65 years | 41/69 | 49/70 | 0.70 | 25/69 | 36/70 | 0.64 |
| | | | (0.46, 1.08) | | | (0.38, 1.08) |

TABLE 3
Analysis on PFS per IRF and OS based on Gender

| | PFS per IRF Subgroup Analysis | | | Overall Survival Subgroup Analysis | | |
|---|---|---|---|---|---|---|
| | Event/N | | Hazard Ratio | Event/N | | Hazard Ratio |
| Gender | A + CHP | CHOP | (95% CI) | A + CHP | CHOP | (95% CI) |
| Male | 59/133 | 80/151 | 0.80 | 32/133 | 49/151 | 0.68 |
| | | | (0.57,1.13) | | | (0.43, 1.06) |
| Female | 36/93 | 44/75 | 0.49 | 19/93 | 24/75 | 0.66 |
| | | | (0.31, 0.78) | | | (0.36, 1.22) |

TABLE 4
Analysis on PFS per IRF and OS based on Baseline ECOG Status

| | PFS per IRF Subgroup Analysis | | | Overall Survival Subgroup Analysis | | |
|---|---|---|---|---|---|---|
| Baseline | Event/N | | Hazard Ratio | Event/N | | Hazard Ratio |
| ECOG Status | A + CHP | CHOP | (95% CI) | A + CHP | CHOP | (95% CI) |
| 0/1 | 76/174 | 105/179 | 0.66 | 34/174 | 61/179 | 0.51 |
| | | | (0.49, 0.89) | | | (0.34, 0.78) |
| 2 | 19/51 | 19/47 | 0.98 | 17/51 | 12/47 | 1.48 |
| | | | (0.51, 1.87) | | | (0.70, 3.11) |

TABLE 5
Analysis on PFS per IRF and OS based on Disease Stage

| | PFS per IRF Subgroup Analysis | | | Overall Survival Subgroup Analysis | | |
|---|---|---|---|---|---|---|
| Disease | Event/N | | Hazard Ratio | Event/N | | Hazard Ratio |
| Stage | A + CHP | CHOP | (95% CI) | A + CHP | CHOP | (95% CI) |
| I or II | 15/42 | 19/46 | 0.95 | 7/42 | 12/46 | 0.66 |
| | | | (0.48, 1.88) | | | (0.25, 1.71) |
| III | 29/57 | 35/67 | 0.69 | 13/57 | 17/67 | 0.71 |
| | | | (0.42, 1.14) | | | (0.33, 1.49) |
| IV | 51/127 | 70/113 | 0.64 | 31/127 | 44/113 | 0.68 |
| | | | (0.45, 0.93) | | | (0.43, 1.07) |

TABLE 6
Analysis on PFS per IRF and OS based on Disease Indication

| | PFS per IRF Subgroup Analysis | | | Overall Survival Subgroup Analysis | | |
|---|---|---|---|---|---|---|
| Disease | Event/N | | Hazard Ratio | Event/N | | Hazard Ratio |
| Indication | A + CHP | CHOP | (95% CI) | A + CHP | CHOP | (95% CI) |
| ALK- | 5/49 | 16/49 | 0.29 | 4/49 | 10/49 | 0.38 |
| positive | | | (0.11, 0.79) | | | (0.12, 1.22) |
| sALCL | | | | | | |
| ALK- | 50/113 | 60/105 | 0.65 | 25/113 | 34/105 | 0.58 |
| negative | | | (0.44, 0.95) | | | (0.35, 0.98) |
| sALCL | | | | | | |
| AITL | 18/30 | 13/24 | 1.40 | 8/30 | 6/24 | 0.87 |
| | | | (0.64, 3.07) | | | (0.29, 2.58) |
| PTCL- | 19/29 | 31/43 | 0.75 | 11/29 | 20/43 | 0.83 |
| NOS | | | (0.41, 1.37) | | | (0.38, 1.80) |

* The Hazard Ratio in the above tables compares clinical benefits of one treatment arm versus another in the clinical trial. A Hazard Ratio of less than 1 means the A + CHP treatment arm provided better clinical benefits than the CHOP treatment arm

### Safety

The duration of treatment was similar between the 2 treatment arms; the median number of weeks of treatment per subject was 18.1 (range, 3, 34) on the A+CHP arm and 18.0 (range, 3 to 31) on the CHOP arm. The median number of cycles received was 6 (range, 1 to 8) for both treatment arms. The median relative dose intensity for brentuximab vedotin was 99.2% (range, 49% to 104%). The median relative dose intensity for vincristine was 99.1% (range, 42% to 116%).

The total incidence of treatment-emergent adverse events (TEAEs), Grade 3 or higher TEAEs, and serious adverse events (SAEs) was similar across the treatment arms (see Table 2). There were fewer Grade 5 TEAEs on the A+CHP arm. The incidence of subjects who discontinued treatment due to an AE was similar across treatment groups (6% and 7% for A+CHP and CHOP, respectively). The only TEAE that resulted in treatment discontinuation for more than one subject in the A+CHP arm was peripheral sensory neuropathy (2 subjects, 1%).

Peripheral neuropathy (PN) occurred at a similar incidence on both arms, was manageable, and resolved over time: Treatment emergent PN was reported for 117 subjects (52%) on the A+CHP arm and 124 subjects (55%) on the CHOP arm. The majority of treatment-emergent PN on both treatment arms was Grade 1. Grade 3 PN occurred in 8 subjects (4%) on the A+CHP arm and 10 subjects (4%) on the CHOP arm. Grade 4 PN occurred in 1 subject on the A+CHP arm (0 subjects in the CHOP arm).

At last follow-up, 102/117 (87%) subjects on the A+CHP arm had complete resolution or residual Grade 1 treatment-emergent PN events, compared with 111/124 subjects (90%) on the CHOP arm. On the A+CHP arm, 15 of 117 subjects (13%) had residual Grade 2 PN and 2 subjects (2%) had residual Grade 3 PN; on the CHOP arm, 12/124 subjects (10%) had residual Grade 2 PN and 1 subject (1%) had residual Grade 3 PN.

The median time to resolution of PN events on the A+CHP arm was 17 weeks (range, 0 to 195) versus 11.4 weeks (range, 0 to 220) on the CHOP arm.

The overall incidence of treatment-emergent febrile neutropenia was similar on both treatment arms (18% versus 15% for A+CHP versus CHOP, respectively) (Table 7). The addition of primary prophylactic G-CSF reduced the incidence and severity to a similar degree in both arms.

TABLE 7
Summary of Neutropenia by Primary Prophylaxis with G-CSF

| | A + CHP | | CHOP | |
|---|---|---|---|---|
| | (N = 223) | | (N = 226) | |
| | No G-CSF | G-CSF | No G-CSF | G-CSF |
| | Primary | Primary | Primary | Primary |
| | Prophylaxis | Prophylaxis* | Prophylaxis | Prophylaxis* |
| | (N = 148) | (N = 75) | (N = 165) | (N = 61) |
| Subjects, n (%) | n (%) | n (%) | n (%) | n (%) |
| Febrile neutropenia in Cycle 1, n (%) | 17 (11) | 9 (12) | 16 (10) | 4 (7) |
| Febrile neutropenia on study, n (%) | 29 (20) | 12 (16) | 26 (16) | 7 (11) |
| Incidence of Grade 3 or higher neutropenia†, n | 67 (45) | 10 (13) | 69 (42) | 8 (13) |
| (%) | | | | |
| Incidence of Grade 4 or higher neutropenia†, n | 39 (26) | 7 (9) | 43 (26) | 6 (10) |
| (%) | | | | |
| Incidence of Grade 3 or higher infections and | 30 (20) | 12 (16) | 23 (14) | 8 (13) |
| infestations (SOC), n (%) | | | | |
| Incidence of serious adverse events of febrile | 41 (28) | 23 (31) | 37 (22) | 15 (25) |
| neutropenia, neutropenia, sepsis, neutropenic | | | | |
| sepsis, pyrexia, or infections and infestations | | | | |
| (SOC), n (%) | | | | |

Results from the trial demonstrated that combination treatment with ADCETRIS plus CHP was superior to the control arm for PFS as assessed by an Independent Review Facility (IRF; hazard ratio=0.71; p-value=0.0110). The ADCETRIS plus CHP arm also demonstrated superior overall survival, a key secondary endpoint, compared with CHOP (hazard ratio=0.66; p-value=0.0244). All other key secondary endpoints, including PFS in patients with systemic anaplastic large cell lymphoma (sALCL), complete remission rate and objective response rate were statistically significant in favor of the ADCETRIS plus CHP arm. The safety profile of ADCETRIS plus CHP in this clinical trial was comparable to CHOP and consistent with the well-established safety profile of ADCETRIS in combination with chemotherapy.

Numerous modifications and variations of the invention as set forth in the above illustrative examples are expected to occur to those skilled in the art. Consequently only such limitations as appear in the appended claims should be placed on the invention.

### REFERENCES

Aaronson N K, Ahmedzai S, Bergman B, Bullinger M, Cull A, Duez N J, Filiberti A, Flechtner H, Fleishman S B, de Haes J C and et al. (1993). J Natl Cancer Inst 85: 365-76.
Cheson B D, Pfistner B, Juweid M E, Gascoyne R D, Specht L, Horning S J, Coiffier B, Fisher R I, Hagenbeek A, Zucca E, Rosen S T, Stroobants S, Lister T A, Hoppe R T, Dreyling M, Tobinai K, Vose J M, Connors J M, Federico M and Diehl V (2007). J Clin Oncol 25: 579-86.
Clopper C J and Pearson E S (1934). The use of confidence or fiducial limits illustrated in the case of the binomial. Biometrika 26: 404-413. Coiffier B, Lepage E, Briere J, Herbrecht R, Tilly H, Bouabdallah R, Morel P, Van Den Neste E, Salles G, Gaulard P, Reyes F, Lederlin P and Gisselbrecht C (2002). N Engl J Med 346: 235-42.
Collett D (1994). Interval-censored survival data. Modelling survival data in medical research. Boca Raton, Fla., Chapman & Hall/CRC: 237-251. Dearden C E, Johnson R, Pettengell R, Devereux S, Cwynarski K, Whittaker S and McMillan A (2011). Br J Haematol 153: 451-85.
Mercadal S, Briones J, Xicoy B, Pedro C, Escoda L, Estany C, Camos M, Colomo L, Espinosa I, Martinez S, Ribera J M, Martino R, Gutierrez-Garcia G, Montserrat E and Lopez-Guillermo A (2008). Ann Oncol 19: 958-63. National Comprehensive Cancer Network (2013). NCCN Guidelines Version 1.2013: Non-Hodgkinis Lymphomas. Available at: http://www.nccn.org. Accessed Feb. 21, 2013.
Pro B, Advani R, Brice P, Bartlett N L, Rosenblatt J D, Illidge T, Matous J, Ramchandren R, Fanale M, Connors J M, Yang Y, Sievers E L, Kennedy D A and Shustov A (2012). J Clin Oncol 30: 2190-6.
Savage K J (2008). Prognosis and primary therapy in peripheral T-cell lymphomas. Hematology Am Soc Hematol Educ Program: 280-8.
Schmitz N, Trumper L, Ziepert M, Nickelsen M, Ho A D, Metzner B, Peter N, Loeffler M, Rosenwald A and Pfreundschuh M (2010). Blood 116: 3418-25.
Shipp M A (1993). N Engl J Med 329: 987-94.
Simon A, Peoch M, Casassus P, Deconinck E, Colombat P, Desablens B, Tournilhac O, Eghbali H, Foussard C, Jaubert J, Vilque J P, Rossi J F, Lucas V, Delwail V, Thyss A, Maloisel F, Milpied N, le Gouill S, Lamy T and Gressin R (2010). Br J Haematol 151: 159-66.
Swerdlow S H, Campo E, Harris N L, Jaffe E S, Pileri S A, Stein H, Thiele J and Vardiman J W (2008). Chapter 11: Mature T- and NK-cell Neoplasms. WHO classification of tumours of haematopoietic and lymphoid tissues. Lyon, France, International Agency for Research on Cancer: 269-319.
Vose J, Armitage J and Weisenburger D (2008). J Clin Oncol 26: 4124-30.
Younes A, Bartlett N L, Leonard J P, Kennedy D A, Lynch C M, Sievers E L and Forero-Torres A (2010). N Engl J Med 363: 1812-21.

1. A method for treating a subject having a mature T cell lymphoma that has exhibited Grade 2 or greater peripheral neuropathy after starting treatment with a combination therapy comprising an anti-CD30 antibody drug conjugate at a dose of 1.8 mg/kg every three weeks in combination with a chemotherapy consisting essentially of cyclophosphamide, doxorubicin and prednisone (CHP), comprising administering anti-CD30 antibody drug conjugate at a dose between 0.9 mg/kg to 1.2 mg/kg.

2. The method of claim 1 wherein when the subject exhibits Grade 3 neuropathy, the administration of anti-CD30 antibody drug conjugate is withheld until peripheral neuropathy decreases to Grade 2 or less and then 0.9 to 1.2 mg/kg anti-CD30 antibody drug conjugate therapy is administered.

3. The method of claim 1 wherein the dose of anti-CD30 antibody drug conjugate is increased to 1.8 mg/kg after the Grade 2 or Grade 3 peripheral neuropathy improves to Grade 1 or less.

4. The method of claim 1 wherein the combination therapy is administered every three weeks.

5. The method of claim 4 wherein the combination therapy is administered on day 1 of a 21 day cycle.

6. The method of claim 4 wherein the combination therapy is administered for no more than six to eight cycles.

7. (canceled)

8. The method of claim 5 wherein the subject receives single-agent anti-CD30 antibody drug conjugate for eight to 10 additional cycles for a total of 16 cycles.

9. (canceled)

10. The method of claim 1 wherein the combination therapy reduces paresthesia, hypoesthesia, polyneuropathy, muscular weakness, and demyelinating polyneuropathy.

11. The method of claim 1 wherein the neuropathy is peripheral motor neuropathy or peripheral sensory neuropathy.

12. (canceled)

13. The method of claim 1 wherein the anti-CD30 antibody drug conjugate is brentuximab vedotin.

14. A method for treating a mature T cell lymphoma in a subject comprising administering a combination therapy comprising an anti-CD30 antibody drug conjugate in combination with a chemotherapy consisting essentially of cyclophosphamide, doxorubicin and prednisone (CHP) and prophylactically administering a granulopoiesis stimulating factor, wherein the granulopoiesis stimulating factor is administered with the initiation of the combination therapy.

15. The method of claim 14 wherein the granulopoiesis stimulating factor is administered from 1 day to 7 days, or from 2 days to 5 days, after the initiation of the combination therapy.

16. The method of claim 14 wherein the granulopoiesis stimulating factor is administered from 1 day to 7 days after a second, or subsequent, administration of the combination therapy, or wherein the granulopoiesis stimulating factor is administered from 2 days to 5 days after a second, or subsequent, administration of the combination therapy.

17. (canceled)

18. The method of claim 14 wherein the granulopoiesis stimulating factor is administered to a subject that has not received an anti-CD30 antibody drug conjugate therapy previously, and/or wherein the subject has not experienced treatment-emergent grade 3-4 neutropenia after administration of the combination therapy.

19. (canceled)

20. The method of claim 14 wherein the anti-CD30 antibody drug conjugate is brentuximab vedotin.

21. A method for reducing the incidence of neutropenia in a subject having mature T cell lymphoma and receiving a combination therapy comprising an anti-CD30 antibody drug conjugate in combination with a chemotherapy consisting essentially of cyclophosphamide, doxorubicin and prednisone (CHP) comprising administering to the subject a granulopoiesis stimulating factor, wherein the granulopoiesis stimulating factor is administered with initiation of the combination therapy.

22. The method of claim 14 wherein the granulopoiesis stimulating factor is a granulocyte-colony stimulating factor (GCSF).

23. The method of claim 22 wherein the GCSF is a long-acting GCSF or a non long-acting GCSF.

24. The method of claim 22,
wherein the GCSF is long-acting GCSF, and is administered 1 day or 2 days after the initiation of the combination therapy, or
wherein the GCSF is not long acting, and is administered 1, 2, 3, 4, 5, 6 or 7 days after the initiation of the combination therapy.

25. (canceled)

26. The method of claim 14 wherein the combination therapy is administered every 3 weeks or every 2 weeks.

27. (canceled)

28. The method of claim 14 wherein the combination therapy is administered on day 1 of a 21 day cycle.

29. The method of claim 28 wherein the combination therapy is administered for no more than six to eight cycles.

30-31. (canceled)

32. The method of claim 1 wherein the anti-CD30 antibody of the anti-CD30 antibody drug conjugate comprises
i) a heavy chain CDR1 set out in SEQ ID NO: 4, a heavy chain CDR2 set out in SEQ ID NO: 6, a heavy chain CDR3 set out in SEQ ID NO: 8; and
ii) a light chain CDR1 set out in SEQ ID NO: 12, a light chain CDR2 set out in SEQ ID NO: 14, and a light chain CDR13 set out in SEQ ID NO: 16.

33. The method of claim 32 wherein the anti-CD30 antibody of the anti-CD30 antibody drug conjugate comprises
i) an amino acid sequence at least 85% identical to a heavy chain variable region set out in SEQ ID NO: 2 and
ii) an amino acid sequence at least 85% identical to a light chain variable region set out in SEQ ID NO: 10.

34. The method of claim 1 wherein the anti-CD30 antibody of the anti-CD30 antibody drug conjugate is a monoclonal anti-CD30 antibody, optionally wherein the anti-CD30 antibody of the anti-CD30 antibody drug conjugate is a chimeric AC10 antibody.

35. (canceled)

36. The method of claim 1 wherein the antibody drug conjugate comprises monomethyl auristatin E and a protease-cleavable linker.

37. The method of claim 36 wherein i) the protease cleavable linker comprises a thiolreactive spacer and a dipeptide; and/or ii) the protease cleavable linker consists of a thiolreactive maleimidocaproyl spacer, a valine-citrulline dipeptide, and a p-amino-benzyloxycarbonyl spacer.

38. (canceled)

39. The method of claim 14 wherein the anti-CD30 antibody drug conjugate is brentuximab vedotin.

40. The method of claim 39 wherein the anti-CD30 antibody drug conjugate is brentuximab vedotin and is administered at 1.8 mg/kg, cyclophosphamide is administered at 750 mg/m², doxorubicin is administered at 50 mg/m², and prednisone is administered at 100 mg on days 1 to 5 of a 21 day cycle.

41. The method of claim 14 wherein the granulopoiesis stimulating factor is administered in a dose range from 5 to 10 mcg/kg/day, or 300 to 600 mcg/day, or 6 mg/dose.

42-43. (canceled)

44. The method of claim 1 wherein the subject is suffering from a mature T cell lymphoma selected from the group consisting of peripheral T cell lymphoma (PTCL), PTCL entities typically manifesting as nodal involvement, angioimmunoblastic T-cell lymphoma, anaplastic large cell lymphomas, peripheral T-cell lymphoma-not otherwise specified, subcutaneous panniculitis-like T-cell lymphoma, hepatosplenic gamma delta T-cell lymphoma, enteropathy-type intestinal T-cell lymphoma, and extranodal T-cell lymphoma-nasal type.

45. (canceled)

46. The method of claim 44 wherein the PTCL is selected from the group consisting of systemic anaplastic large cell lymphoma (sALCL), angioimmunoblastic T-cell lymphoma (AITL), peripheral T-cell lymphoma-not otherwise specified (PTCL-NOS), Adult T-Cell Leukemia/Lymphoma (ATLL), Enteropathy-associated T-cell lymphoma (EATL) and Hepatosplenic T-cell lymphoma; or
wherein the PTCL is selected from the group consisting of angioimmunoblastic T-cell lymphoma (AITL), peripheral T-cell lymphoma-not otherwise specified (PTCL-NOS), Adult T-Cell Leukemia/Lymphoma (ATLL), Enteropathy-associated T-cell lymphoma (EATL) and Hepatosplenic T-cell lymphoma.

47. (canceled)

48. The method of claim 46 wherein the sALCL is selected from the group consisting of anaplastic lymphoma kinase positive (ALK+) sALCL and anaplastic lymphoma kinase negative (ALK−) sALCL.

49-53. (canceled)

54. The method of claim 44, wherein the subject has an International Prognostic Index (IPI) score 2 and/or wherein the subject has not previously been treated for a hematoloqic cancer or wherein the subject has previously been treated for a hematoloqic cancer and the cancer has relapsed or is refractory.

55-56. (canceled)

57. The method of claim 44, wherein the PTCL
i) is a stage Ill or stage IV PTCL;
ii) is a CD30-expressing PTCL; and/or
iii) is a CD30-expressing PTCL.

58-60. (canceled)

61. The method of claim 44 wherein
i) when the mature T cell lymphoma is PTCL, and wherein if the subject is diagnosed with Grade 2 or greater peripheral motor neuropathy after starting treatment with a combination therapy comprising an anti-CD30 antibody drug conjugate at a dose of 1.8 mg/kg every three weeks in combination with a chemotherapy consisting essentially of cyclophosphamide, doxorubicin and prednisone (CHP), the dose of anti-CD30 antibody drug conjugate is reduced to 1.2 mg/kg;
ii) when the mature T cell lymphoma is PTCL, and wherein if the subject is diagnosed with Grade 3 or greater peripheral sensory neuropathy after starting treatment with a combination therapy comprising an anti-CD30 antibody drug conjugate at a dose of 1.8 mg/kg every three weeks in combination with a chemotherapy consisting essentially of cyclophosphamide, doxorubicin and prednisone (CHP), the dose of anti-CD30 antibody drug conjugate is reduced to 1.2 mg/kg.

62. (canceled)

63. A method for decreasing the incidence of infection in a subject having mature T cell lymphoma and receiving a combination therapy comprising an anti-CD30 antibody drug conjugate in combination with a chemotherapy consisting essentially of cyclophosphamide, doxorubicin and prednisone (CHP) comprising administering to the subject a granulopoiesis stimulating factor in an amount effective to reduce infections, wherein the granulopoiesis stimulating factor is administered with the initiation of the combination therapy.

64. The method of claim 63 wherein the granulopoiesis stimulating factor is administered from 1 day to 7 days after, or from 2 days to 5 days, after the initiation of the combination therapy.

65. The method of claim 63 wherein the granulopoiesis stimulating factor is administered from 1 day to 7 days after a second, or subsequent, administration of the combination therapy or wherein the granulopoiesis stimulating factor is administered from 2 days to 5 days after the second, or subsequent, administration of the combination therapy.

66-68. (canceled)

69. The method of claim 63 wherein the granulopoiesis stimulating factor is a granulocyte-colony stimulating factor GCSF.

70-78. (canceled)

79. The method of claim 63 wherein the anti-CD30 antibody of the anti-CD30 antibody drug conjugate comprises
i) a heavy chain CDR1 set out in SEQ ID NO: 4, a heavy chain CDR2 set out in SEQ ID NO: 6, a heavy chain CDR3 set out in SEQ ID NO: 8; and
ii) a light chain CDR1 set out in SEQ ID NO: 12, a light chain CDR2 set out in SEQ ID NO: 14, and a light chain CDR13 set out in SEQ ID NO: 16.

80. The method of claim 79 wherein the anti-CD30 antibody of the anti-CD30 antibody drug conjugate comprises
i) an amino acid sequence at least 85% identical to a heavy chain variable region set out in SEQ ID NO: 2 and
ii) an amino acid sequence at least 85% identical to a light chain variable region set out in SEQ ID NO: 10.

81. The method of claim 63 wherein the anti-CD30 antibody of the anti-CD30 antibody drug conjugate is a monoclonal anti-CD30 antibody, optionally wherein the anti-CD30 antibody of the anti-CD30 antibody drug conjugate is a chimeric AC10 antibody.

82. (canceled)

83. The method of claim 63 wherein the antibody drug conjugate comprises monomethyl auristatin E and a protease-cleavable linker.

84-85. (canceled)

86. The method of claim 63 wherein the anti-CD30 antibody drug conjugate is brentuximab vedotin.

87-107. (canceled)

108. The method of claim 86, wherein the granulopoiesis stimulating factor is administered from 1 to 8 days after initiation of the combination therapy.


SEQUENCE LISTING (16 sequences)

SEQ ID NO: 1 - DNA, 351 bp, Mus musculus
  source 1..351: mol_type = genomic DNA
  CDS 1..351
cagatccagctgcagcagtctggacctgaggtggtgaagcctggggcttcagtgaagatatcctgcaaggcttctggctacaccttcactgactactatataacctgggtgaagcagaagcctggacagggacttgagtggattggatggatttatcctggaagcggtaatactaagtacaatgagaagttcaagggcaaggccacattgactgtagacacatcctccagcacagccttcatgcagctcagcagcctgacatctgaggacactgctgtctatttctgtgcgaactatggtaactactggtttgcttactggggccaagggactcaggtcactgtctctgca

SEQ ID NO: 2 - protein, 117 aa, Mus musculus
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 3 - DNA, 15 bp, Mus musculus
  source 1..15: mol_type = genomic DNA
gactactatataacc

SEQ ID NO: 4 - protein, 5 aa, Mus musculus
DYYIT

SEQ ID NO: 5 - DNA, 51 bp, Mus musculus
  source 1..51: mol_type = genomic DNA
tggatttatcctggaagcggtaatactaagtacaatgagaagttcaagggc

SEQ ID NO: 6 - protein, 17 aa, Mus musculus
WIYPGSGNTKYNEKFKG

SEQ ID NO: 7 - DNA, 24 bp, Mus musculus
  source 1..24: mol_type = genomic DNA
tatggtaactactggtttgcttac

SEQ ID NO: 8 - protein, 8 aa, Mus musculus
YGNYWFAY

SEQ ID NO: 9 - DNA, 333 bp, Mus musculus
  source 1..333: mol_type = genomic DNA
  CDS 1..333
gacattgtgctgacccaatctccagcttctttggctgtgtctctagggcagagggccaccatctcctgcaaggccagccaaagtgttgattttgatggtgatagttatatgaactggtaccaacagaaaccaggacagccacccaaagtcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtggacgttcggtggaggcaccaagctggaaatcaaa

SEQ ID NO: 10 - protein, 111 aa, Mus musculus
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 11 - DNA, 45 bp, Mus musculus
  source 1..45: mol_type = genomic DNA
aaggccagccaaagtgttgattttgatggtgatagttatatgaac

SEQ ID NO: 12 - protein, 15 aa, Mus musculus
KASQSVDFDGDSYMN

SEQ ID NO: 13 - DNA, 21 bp, Mus musculus
  source 1..21: mol_type = genomic DNA
gctgcatccaatctagaatct

SEQ ID NO: 14 - protein, 7 aa, Mus musculus
AASNLES

SEQ ID NO: 15 - DNA, 27 bp, Mus musculus
  source 1..27: mol_type = genomic DNA
cagcaaagtaatgaggatccgtggacg

SEQ ID NO: 16 - protein, 9 aa, Mus musculus
QQSNEDPWT